(19) United States
(12) Patent Application Publication
(10) Pub. No.: US 2025/0263772 A1
(43) Pub. Date: Aug. 21, 2025

(54) TEST METHOD FOR PHYSICAL FRAILTY, TEST REAGENT FOR PHYSICAL FRAILTY, METHOD FOR SCREENING THERAPEUTIC CANDIDATE SUBSTANCE FOR PHYSICAL FRAILTY, TEST MELTHOD FOR MENTAL FRAILTY, TEST REAGENT FOR MENTAL FRAILTY, AND METHOD FOR SCREENING THERAPEUTIC CNADIDATE SUBSTANCE FOR MENTAL FRAILTY

(71) Applicant: Bioradical Research Institute Corp., Yokohama-shi, Kanagawa (JP)

(72) Inventors: Masaichi Lee, Yokohama-shi, Kanagawa (JP); Tomoko Komatsu, Yokohama-shi, Kanagawa (JP)

(73) Assignee: Bioradical Research Institute Corp., Yokohama-shi, Kanagawa (JP)

(21) Appl. No.: 18/684,141

(22) PCT Filed: Jun. 24, 2022
(86) PCT No.: PCT/JP2022/025280

§ 371 Date: May 10, 2024

(30) Foreign Application Priority Data
Aug. 18, 2021 (JP) 2021-133689

(51) Int. Cl. C12Q 1/26; G01N 33/92

(52) U.S. Cl. CPC C12Q 1/26; G01N 33/92; G01N 2333/90283; G01N 2800/2814; G01N 2800/52

(57) ABSTRACT

The present invention provides a test method capable of detecting physical frailty and a measurement reagent for use in the test method. A test method for physical frailty of the present invention includes: measuring radical scavenging ability in a biological sample of a subject.

### TECHNICAL FIELD

The present invention relates to a test method for physical frailty, a test reagent for physical frailty, a method for screening a therapeutic candidate substance for physical frailty, a test method for mental frailty, a test reagent for mental frailty, and a method for screening a therapeutic candidate substance for mental frailty.

### BACKGROUND ART

With the aging society, the number of frailty patients who are at an intermediate stage between a healthy state and a state requiring nursing is increasing. The frailty includes physical frailty, mental frailty, and the like. The physical frailty is associated with sarcopenia caused by decreased muscle strength (Non Patent Literature 1).

### CITATION LIST

### Non Patent Literature

Non Patent Literature 1: “Significance of Frailty,” [online], Japanese Journal of Geriatrics, Searched on Aug. 18, 2021<https://www.jpn-geriat-soc.or.jp/publications/other/pdf/review_51_6_497.pdf>

### SUMMARY OF INVENTION

### Technical Problem

Regarding sarcopenia, by intervening in a state of physical frailty, it is possible to suppress the progression rate to a state requiring nursing. Therefore, there is a need for a test method capable of detecting physical frailty.

With the foregoing in mind, an object of the present invention is to provide a test method capable of detecting physical frailty and a measurement reagent for use in the test method.

### Solution to Problem

In order to achieve the above objective, the present invention provides a test method for physical frailty (also referred to as “first test method” hereinafter), including: measuring radical scavenging ability in a biological sample of a subject.

The present invention also provides a test reagent for physical frailty (also referred to as “first test reagent” hereinafter), including: a reagent for measuring radical scavenging ability.

The present invention also provides a method for screening a therapeutic candidate substance for physical frailty (also referred to as “first screening method” hereinafter), including: selecting, from test substances, an activating substance that reduces radical scavenging ability as a therapeutic candidate substance for physical frailty (also referred to as “first selecting” hereinafter).

The present invention also provides a detection method for radical scavenging ability in a subject who has a suspected case of physical frailty (also referred to as “first detection method” hereinafter), including: detecting radical scavenging ability in a biological sample of the subject using a reagent for measuring radical scavenging ability (also referred to as “first detecting” hereinafter).

The present invention also provides a test method for mental frailty (also referred to as “second test method” hereinafter), including: measuring radical scavenging ability in a biological sample of a subject; and measuring superoxide dismutase (SOD) activity in the biological sample of the subject (also referred to as “second measuring” hereinafter).

The present invention also provides a test kit for mental frailty (also referred to as “test kit” hereinafter), including: a reagent for measuring radical scavenging ability; and a reagent for measuring SOD activity.

The present invention also provides a method for screening a therapeutic candidate substance for mental frailty (also referred to as “second screening method” hereinafter), including: selecting, from test substances, an activating substance that reduces radical scavenging ability and improves SOD activity as a therapeutic candidate substance for mental frailty (also referred to as “second selecting” hereinafter).

The present invention also provides a detection method for radical scavenging ability and SOD activity in a subject who has a suspected case of mental frailty (also referred to as “second detection method” hereinafter), including: detecting radical scavenging ability in a biological sample of the subject using a reagent for measuring radical scavenging ability (first detecting); and detecting SOD activity in the biological sample of the subject using a reagent for measuring SOD activity (second detecting).

### Advantageous Effects of Invention

The present invention can detect whether or not a subject has physical frailty by using a biological sample derived from the subject.

### BRIEF DESCRIPTION OF DRAWINGS

FIG. 1 is a graph showing the amounts of saliva of the subjects in Example 1.

FIG. 2 is a graph showing the average amount of saliva of each subject in each age group in Example 1.

FIGS. 3A and 3B are graphs showing the maximum grip strength values of the subjects in Example 1. FIG. 3A shows the results of the maximum grip strength values of the subjects, and FIG. 3B shows the results of the average of the maximum grip strength values of the subjects.

FIG. 4 is a graph showing the maximum grip strength value of the subjects in each age group and the average of the maximum grip strength values of the subjects in each age group in Example 1.

FIG. 5 is a graph showing the results of ESR in the saliva samples, showing the results of i-STrap of the subjects in Example 1.

FIG. 6 is a graph showing the results of ESR in the saliva samples, showing the results of the average of i-STrap values of the subjects in each age group in Example 1.

FIGS. 7A and 7B are graphs showing the correlation between the grip strength and the radical scavenging ability of the subjects in Example 1.

FIGS. 8A and 8B are graphs showing the correlation between the grip strength and the radical scavenging ability of the subjects aged 50 years or younger in Example 1.

FIGS. 9A and 9B are graphs showing the correlation between the grip strength and the radical scavenging ability of the subjects aged 50 to 70 years in Example 1.

FIGS. 10A and 10B are graphs showing the correlation between the grip strength and the radical scavenging ability of the subjects aged 70 years or older in Example 1.

FIG. 11 is a graph showing the amounts of saliva of the subjects in Example 2.

FIG. 12 is a diagram showing 12 absorption lines (peaks) originating from the internal magnetic field of ¹⁴N and ¹H at the β and γ positions in the Electron Spin Resonance (ESR) spectrum in Example 2.

FIG. 13 is a graph showing the ESR results for the saliva samples in Example 2.

### DESCRIPTION OF EMBODIMENTS

### Definition

As used herein, the “testing for physical frailty” refers to, for example, detection of physical frailty, determination of physical frailty, screening for physical frailty, determination of a physical frailty preventive effect, determination of a physical frailty therapeutic effect, determination of a physical frailty patient who responds to a therapeutic agent, determination of a therapeutic agent that takes an effect on an individual physical frailty patient, a test method for diagnosing physical frailty, or a test for treating physical frailty. The “determination of physical frailty” may be, for example, determining whether physical frailty occurs; testing, detection or diagnosis of physical frailty; determination, testing, detection, or diagnosis of the likelihood (risk) of having physical frailty; prediction of prognosis after treatment of physical frailty; or determination of the therapeutic effect of a therapeutic agent for physical frailty, and it may be replaced with any of these.

As used herein, the “testing for mental frailty” refers to, for example, detection of mental frailty, determination of mental frailty, screening for mental frailty, determination of a mental frailty preventive effect, determination of a mental frailty therapeutic effect, determination of a mental frailty patient who responds to a therapeutic agent, determination of a therapeutic agent that takes an effect on an individual mental frailty patient, a test method for diagnosing mental frailty, or a test for treating mental frailty. The “determination of mental frailty” may be, for example, determining whether mental frailty occurs; testing, detection or diagnosis of mental frailty; determination, testing, detection, or diagnosis of the likelihood (risk) of having mental frailty; prediction of prognosis after treatment of mental frailty; or determination of the therapeutic effect of a therapeutic agent for mental frailty, and it may be replaced with any of these.

In the present invention, “having a disease” may refer to the state of being affected with the disease or to develop the disease.

As used herein, the term “treatment” refers to therapeutic treatment and/or prophylactic treatment. As used herein, the term “treatment” means the treatment, cure, prevention, suppression, amelioration, or improvement of a disease, condition, or disorder, or stop, suppression, reduction, or delay of the progression of a disease, condition, or disorder. As used herein, the term “prevention” refers to a decrease in the likelihood of developing a disease or condition, or a delay in the offset of a disease or condition. The term “treatment” may be, for example, treatment for a patient who develops a target disease, or treatment of a model animal with a target disease.

As used herein, the “frailty” refers to various function decline syndromes (decreased reserve abilities) associated with aging, which is a condition with increased vulnerability to diverse emerging health problems. The frailty” is generally used as a concept including a physical problem and/or a mental problem.

As used herein, the term “physical frailty” is included in the frailty” and refers to a state that is susceptible to a health problem due to a decrease in physical strength and muscle strength associated with aging. The physical frailty can be assessed, for example, using a combination of items such as ambulation, muscle strength, cognitive function, nutritional status, balance ability, endurance, physical activity, social activity, and the like. For the diagnostic criteria for physical frailty, for example, Cardiovascular Health Study (CHS index), Frailty index, or the like can be used. When using the CHS index, the physical frailty is assessed using, as representative signs of the frailty, the five items of (1) unintentional weight loss, (2) exhaustion (low-energy condition, fatigability), (3) low levels of activity (physical activities in everyday life), (4) slowness while walking (decreased physical performance), and (5) muscle (grip strength) weakness in a subject, as summarized in Table 1 below. In the CHS index, when three of the five items apply, the subject can be assessed as having frailty, and when one or two of the five items apply, the subject can be assessed as having pre-frailty (pre-physical frailty). In the present invention, the physical frailty may be assessed using a combination of other assessment methods, for example. Examples of the other assessment methods include Frail scale, Edmonton Frail scale (EFS), and Tilburg Frailty Indicator (TFI).

TABLE 1

| Item | Definition | | | |
|---|---|---|---|---|
| (1) Unintentional | Loss of more than 4.5 kg unintentionally in past year | | | |
| weight loss | | | | |
| (2) Exhaustion | Self-report | | | |
| | (i) felt more tired than usual since last month | | | |
| | (ii) felt weak in past month | | | |
| (3) Low levels | Assessment of physical activities in everyday life | | | |
| of activity | (Assessment of physical activities of recreational | | | |
| | activities, etc.) | | | |
| (4) Slowness | Female | 159 cm ≤ height 7 seconds or longer | | |
| while walking | | 159 cm > height 6 seconds or longer | | |
| 15 feet (4.57 m) | Male | 173 cm ≤ height 7 seconds or longer | | |
| | | 173 cm > height 6 seconds or longer | | |
| (5) Muscle (grip | Female | BMI ≤ 23 | ≤17 | kg |
| strength) | | 23.1 ≤ BMI ≤ 26 | ≤17.3 | kg |
| weakness | | 26.1 ≤ BMI ≤ 29 | ≤18 | kg |
| | | 29 < BMI | ≤21 | kg |
| | Male | BMI ≤ 24 | ≤29 | kg |
| | | 24.1 ≤ BMI ≤ 26 | ≤30 | kg |
| | | 26.1 ≤ BMI ≤ 28 | ≤30 | kg |
| | | 28 < BMI | ≤32 | kg |

In the present invention, “mild cognitive impairment” refers to diseases classified as ICD code F06.7 (mild cognitive impairment) in the International Classification of Diseases 11th Revision (ICD11), for example. The MCI can be assessed using MoCA-J (Japanese version of MoCA), for example, and, when the score is 25 points or less, a subject can be assessed as having MCI. In the present invention, MCI may be assessed using a combination of other assessment methods, for example. Examples of the other assessment methods include Trail Making Test Japanese version (TMT-J), Clinical Dementia Rating (CDR), Functional Assessment Staging (FAST), Hasegawa's Dementia Scale-Revised (HDS-R), Mini-Mental State Examination (MMSE), and DASC-21 (Dementia Assessment Sheet in Community-based Integrated Care System).

As used herein, the term “mental frailty” refers to a state of occurring mild cognitive impairment as cognitive decline progresses in addition to having physical frailty.

As used herein, the term “radical” refers to an atom, molecule, or ion having unpaired electrons.

As used herein, the term “antioxidant” refers to a substance that captures reactive oxygen species. Examples of the reactive oxygen species include the radical species (radicals); and non-radical species such as singlet oxygen (¹O₂), ozone (O₃), and hydrogen peroxide (H₂O₂).

As used herein, the term “radical scavenging ability” refers to an ability to capture radicals by a test substance.

### <Physical Frailty Marker>

In one aspect, the present invention discloses a marker that is an indicator of physical frailty. A physical frailty marker of the present disclosure is radical scavenging ability. The physical frailty marker of the present disclosure is radical scavenging ability, and there is no particular limitation on other constituents or conditions.

As a result of intensive studies, the inventors of the present invention found that the radical scavenging ability in a living organism, in particular, the radical scavenging ability by antioxidants in saliva, correlates with the development of physical frailty, thereby establishing the present invention. According to the present invention, the likelihood of subjects having physical frailty (morbidity risk) and the like can be tested by measuring the radical scavenging ability. Also, in the present disclosure, because the radical serves as a target with respect to physical frailty, therapeutic candidate substances for physical frailty can be obtained through screening by using the target. Therefore, the present invention is extremely useful in the clinical and biochemical fields.

Examples of the radical include a hydroxyl radical (·OH), an alkoxy radical (LO·), a peroxy radical (LOO·), a hydroperoxy radical (HOO·), nitric oxide (NO·), nitrogen dioxide (NO₂·), a superoxide anion (O²⁻), and a lipid radical.

The antioxidant may, for example, capture any one of or two or more of the reactive oxygen species. The reactive oxygen species capturing can also be referred to as, for example, reactive oxygen species scavenging. The radical capturing can also be referred to as, for example, radical scavenging. The reactive oxygen species capturing may be performed, for example, by the antioxidant donating a hydrogen atom to the reactive oxygen species and converting the reactive oxygen species into other more stable molecules (e.g., water). The antioxidant can also be referred to as, for example, a capturing substance of reactive oxygen species, radical species, or singlet oxygen, or a scavenging substance of reactive oxygen species, radical species, or singlet oxygen. In addition, the antioxidant can suppress or prevent oxidation of other molecules also present, by reactive oxygen species, for example.

The radical scavenging ability can be assessed by a method of assessing the antioxidant ability using i-STrap method in accordance with Example 1 described below. Specifically, in the assessment of the antioxidant ability using i-STrap method, radicals such as tBuO·(tert-butyloxyl radicals) and tBu (tert-butyl radicals) can be assessed using, as a reference value, an ESR signal of electron spin resonance (ESR) spin adduct (DPhPMPO-OOtBu, DPhPMPO-OtBu, DPhPMPO-tBu) spin-trapped by 2-diphenylphosphinoyl-2-methyl-3,4-dihydro-2H-pyrrole N-oxide (DPhPMPO). As a specific example, when the saliva having an antioxidant action is also present with the radical, the ESR signal is decreased as compared to the reference value, and thus the radical scavenging ability can be assessed. Therefore, the radical scavenging ability can be assessed by setting an i-STrap value in a reaction system to which a (saliva) sample is not added to 1 (reference value), and calculating the difference (1-R) between the reference value and an i-STrap value (R) in a reaction system to which a (saliva) sample is added. In the method for assessing the antioxidant ability, when the difference (1-R) between the reference value and the measurement value obtained using the test substance is, for example, 0.1 or more, 0.15 or more, 0.2 or more, 0.21 or more, 0.22 or more, 0.23 or more, 0.24 or more, 0.25 or more, 0.26 or more, 0.27 or more, 0.28 or more, 0.29 or more, 0.30 or more, 0.31 or more, 0.32 or more, 0.33 or more, 0.34 or more, 0.35 or more, 0.36 or more, 0.37 or more, 0.38 or more, 0.39 or more, or 0.4 or more, the test substance can be assessed as having radical scavenging ability.

The radical scavenging ability can be used as a physical frailty marker and can be particularly suitably used as a physical frailty marker caused by sarcopenia, locomotive syndrome, depression, forgetfulness, mild dementia, dementia, gait disorder, fall, and joint contracture.

The radical scavenging ability is preferably the lipid radical scavenging ability. The radical scavenging ability in the living organism is presumed to be caused by an antioxidant contained in the living organism. Therefore, in the present invention, the antioxidant may be used as a physical frailty marker in addition to or in place of the radical scavenging ability.

The physical frailty marker may also be used as a marker for diagnosing or detecting physical frailty, a marker for predicting a prognosis of physical frailty, or a marker for predicting or determining a physical frailty therapeutic effect, for example, as described below.

### Physical Frail Test Method

In another aspect, the present invention discloses a method for testing the likelihood of physical frailty. The test method for physical frailty of the present disclosure includes measuring radical scavenging ability in a biological sample of a subject, as described above. The first test method of the present disclosure includes measuring, as a physical frailty marker, the radical scavenging ability in the biological sample of the subject, and there is no particular limitation on other steps or conditions. Regarding the first test method of the present disclosure, reference can be made to the description as to the physical frailty marker of the present disclosure.

With the first test method of the present disclosure, it is possible to perform testing for the likelihood of physical frailty, for example. The “testing for physical frailty” refers to detection of physical frailty, determination of physical frailty, screening for physical frailty, determination of a physical frailty preventive effect, determination of a physical frailty therapeutic effect, determination of a physical frailty patient who responds to a therapeutic agent, determination of a therapeutic agent that takes an effect on an individual physical frailty patient, a test method for diagnosing physical frailty, or a test for treating physical frailty. The “determination of physical frailty” may be, for example, determining whether physical frailty occurs; testing, detection or diagnosis of physical frailty; determination, testing, detection, or diagnosis of the likelihood (risk) of having physical frailty; prediction of prognosis after treatment of physical frailty; or determination of the therapeutic effect of a therapeutic agent for physical frailty.

Examples of the subject include humans and non-human animals other than humans. As described above, examples of the non-human animals include mammals such as mice, rats, dogs, monkeys, rabbits, sheep, and horses; birds; and fish.

There is no particular limitation on the type of the biological sample, and examples thereof include body fluids, body fluid-derived cells, organs, tissues, and cells that are isolated from living organisms. Examples of the body fluid include blood; saliva; urine; lymphatic fluid; synovial fluid of joints; and cerebrospinal fluids such as bone marrow aspirate and cerebrospinal fluid. Specific examples of the blood include whole blood, serum, and plasma. Examples of the body fluid-derived cells include blood-derived cells, and specific examples thereof include blood cells such as blood cells, leukocytes, and lymphocytes. Further, with the physical frailty marker, for example, physical frailty of a subject can be tested by the radical scavenging ability in saliva. Therefore, the biological sample is preferably saliva because it is possible to reduce the burden on patients and doctors, for example.

The radical scavenging ability to be measured is, for example, lipid radical scavenging ability. The radical scavenging ability may be measured, for example, by measuring the lipid radical scavenging ability for the biological sample, or it may be measured by measuring the radical scavenging ability of the antioxidant for the antioxidant obtained from the biological sample after extraction, rough purification, or purification, for example. There is no particular limitation on the method for measuring the lipid radical scavenging ability of the antioxidant, and a known method can be adopted. Specific examples of the method for measuring the lipid radical scavenging ability of the antioxidant include the Electron Spin Resonance (ESR) spin trapping method (i-STrap method) using tert-butyl hydroperoxide (tBuOOH) and hemoglobin as a lipid radical generating agent and 2-diphenylphosphinoyl-2-methyl-3,4-dihydro-2H-pyrrole N-oxide (DPhPMPO) as a spin trapping agent; the Oxygen Radical Absorbance Capacity (ORAC) method; and the Total Radical-Trapping Antioxidant Parameter (TRAP) method for peroxyl radicals, and an i-STrap method is preferred because it is possible to suppress the effects of other reactive oxygen species such as hydrogen peroxide, hydroxyl radicals, and singlet oxygen and to specifically detect radicals, particularly lipid radicals, and the i-STrap method has good sensitivity. The radical scavenging ability may be measured qualitatively or quantitatively. When the radical scavenging ability is measured quantitatively, the radical scavenging ability may be referred to as a radical scavenging ability value.

The first test method of the present disclosure further includes, for example, testing the likelihood of the subject having physical frailty by comparing the radical scavenging ability value in the biological sample of the subject (also referred to as a “test biological sample” hereinafter) with a threshold (also referred to as “first testing” hereinafter). The threshold is not particularly limited, and may be, for example, a threshold calculated from radical scavenging ability values in biological samples of healthy subjects, radical scavenging ability values in biological samples of physical frailty patients, or the radical scavenging ability values in the biological samples of the healthy subjects and the physical frailty patients. In the case of prognostic assessment, the threshold may be the radical scavenging ability value obtained before or after (e.g., immediately after) treating the same subject, for example.

The threshold can be obtained using biological samples (also referred to as “reference biological samples” hereinafter) isolated from healthy subjects and/or physical frailty patients as described above, for example. Further, reference biological samples are isolated from multiple healthy subjects and multiple physical frailty patients, the radical scavenging ability thereof is measured, and then the threshold may be calculated statistically, for example based on the obtained radical scavenging ability. In this case, as the threshold, for example, clinical diagnosis values such as a diagnosis threshold (cut-off value), a treatment threshold, a preventive medicine threshold, and the like can be used. Also, in the case of prognostic assessment, a reference biological sample isolated from the same subject after treatment may be used, for example. The threshold may be measured at the same time as the measurement for the test biological sample from the subject or may be measured in advance, for example. The latter case is preferable because there is no need to obtain a threshold each time a test biological sample from the subject is measured, for example. It is preferable that the test biological sample from the subject and the reference biological sample are collected under the same conditions, and the radical scavenging abilities therein are measured under the same conditions, for example.

There is no particular limitation on the method for assessing the likelihood of a subject having physical frailty in the first testing, and the assessment method can be determined as appropriate according to the type of threshold. As specific examples, when the radical scavenging ability value in the test biological sample from the subject is the same as the radical scavenging ability values in the reference biological samples of the healthy subjects (when there is no significant difference), when the radical scavenging ability value in the test biological sample from the subject is significantly lower than the radical scavenging ability values in the reference biological samples of the healthy subjects, and/or when the radical scavenging ability value in the test biological sample from the subject is significantly lower than the threshold calculated from the radical scavenging ability values in the reference biological samples of healthy subjects and the physical frailty patients, the subject is assessed as having no or little likelihood (also referred to as “risk” or “degree of risk,” the same applies, hereinafter) of having physical frailty. Further, when the radical scavenging ability value in the test biological sample from the subject is significantly higher than the radical scavenging ability values in the reference biological samples of the healthy subjects, when the radical scavenging ability value in the test biological sample from the subject is the same as the radical scavenging ability values in the reference biological samples of the physical frailty patients (when there is no significant difference), when the radical scavenging ability value in the test biological sample from the subject is significantly higher than the radical scavenging ability values in the reference biological samples of the physical frailty patients, and/or when the radical scavenging ability value in the test biological sample from the subject is significantly higher than the threshold calculated from the radical scavenging ability values in the reference biological samples of the healthy subjects and the physical frailty patients, the subject can be assessed as likely, or highly likely (having high risk), to have physical frailty. Also, in the testing, the level of physical frailty can be assessed by comparing the radical scavenging ability value in the test biological sample from the subject with the radical scavenging ability values in the reference biological samples of the physical frailty patients at respective levels of cognitive impairment. Specifically, when the test biological sample from the subject has a radical scavenging ability value similar to that in the reference biological samples at a certain level of cognitive impairment (when there is no significant difference), for example, the subject can be assessed as likely to or highly likely to have cognitive impairment at that level.

When the prognostic state is assessed in the first testing, an assessment may be made in the same manner as the above-described manner or can be assessed using the radical scavenging ability value in a reference biological sample obtained after treating the same subject, for example. As specific examples, when the radical scavenging ability value in the test biological sample from the subject is the same as the threshold (when there is no significant difference), and/or when the radical scavenging ability value in the test biological sample from the subject is significantly lower than the threshold, the subject can be assessed as having no or little likelihood of a relapse of physical frailty after the treatment. Also, when the radical scavenging ability value in the test biological sample from the subject is significantly higher than the threshold, the subject can be assessed as likely to or highly likely to relapse or worsen after the treatment.

In the first testing, biological samples may be collected from the same subject over time, and the radical scavenging ability values in the biological samples may be compared to each other, for example. As a result, in the testing, when the radical scavenging ability decreases over time, it can be determined that the likelihood of having physical frailty has decreased or the patient has been cured, and, when the radical scavenging ability increases over time, it can be determined that the likelihood of having physical frailty has increased, for example.

In the first test method according to the present disclosure, treatment may be administered to the subject based on the results of the first testing, for example. Specifically, the first test method according to the present disclosure may include administering a therapeutic agent for physical frailty to a subject who has received a test result indicating that the subject has physical frailty in the first testing, for example. Conditions such as an administration form, an administration period, a dosage, and an administration interval of the therapeutic agent for physical frailty and the like can be set as appropriate according to the type of the therapeutic agent for physical frailty. The therapeutic agent for physical frailty may be a therapeutic candidate substance obtained through the screening method described below.

While the radical scavenging ability is used as the physical frailty indicator (marker) in the first test method of the present disclosure, the antioxidant may be used instead of or in addition to the radical scavenging ability in the first test method of the present disclosure. In this case, in the test method of the present disclosure, reference can be made to the above descriptions by replacing the “radical scavenging ability” with “antioxidant” and replacing the “radical scavenging ability value” with “amount of antioxidant.”

The subject is not particularly limited, and may be, for example, a physical frailty patient, a subject who is unknown to have physical frailty, or a subject who has a suspected case of physical frailty. The subject may be a mild cognitive impairment (MCI) subject. In this case, the test method of the present disclosure can test the likelihood of mental frailty, and reference can be made to the description as to the physical frailty by replacing the “physical frailty” with “mental frailty.” When the subject is a human, the subject is preferably aged 50 years or older and under 70 years.

While the assessment is made for the “physical frailty” in the first test method of the present disclosure, the present disclosure is not limited thereto, and the assessment may be made for the “frailty” instead of the “physical frailty.” In this case, in the first test method of the present disclosure, reference can be made to the description as to the physical frailty by replacing the “physical frailty” with the “frailty.”

As described above, the first test method according to the present disclosure can be used as a method for diagnosing or detecting physical frailty, a method for predicting the prognosis of physical frailty, and a method for predicting or determining effects of physical frailty treatment, for example. Therefore, the first test method according to the present disclosure can also be used as a companion diagnostic method for selecting patients (responders) who respond to the therapeutic agent for physical frailty and adjusting the dosage of the therapeutic agent for physical frailty.

### Test Reagent

In another aspect, the present invention discloses a test reagent capable of testing for the likelihood of physical frailty. As described above, the test reagent according to the present disclosure contains a reagent for measuring radical scavenging ability. The test reagent according to the present disclosure contains a reagent for measuring radical scavenging ability, and there is no particular limitation on other constituents or conditions. With the test reagent of the present invention, the test method according to the present invention can be easily performed. Regarding the test reagent of the present disclosure, reference can be made to the descriptions as to the physical frailty marker and the test method of the present invention.

The reagent for measuring radical scavenging ability can be appropriately determined according to, for example, a method for measuring the radical scavenging ability. Specifically, when the radical scavenging ability is measured by the ESR method such as the i-STrap method, examples of the reagent for measuring radical scavenging ability include a radical generating agent and a radical detecting agent. The radical generating agent may include a lipid radical generating agent, and the radical detecting agent may include a lipid radical detecting agent. Examples of the radical generating agent include a combination of tert-butyl hydroperoxide (tBuOOH) and hemoglobin. Examples of the radical detecting agent include 2-diphenylphosphinoyl-2-methyl-3,4-dihydro-2H-pyrrole N-oxide (DPhPMPO), N-tert-Buthyl-α-Phenylnitrone (PBN), α-(4-Pyridyl-1-Oxide)-N-tert-Butylnitrone (4-POBN), 3,3,5,5-Tetramethyl-1-Pyrroline-N-Oxide (M4PO), 3,5-Dibromo-4-Nitrosobenzenesulfonic Acid, Sodium Salt (DBNBS), 3,5-Dibromo-4-Nitrosobenzenesulfonic Acid-d2, and Sodium Salt (DBNBS-d2).

As described above, with the test reagent according to the present disclosure, the first test method according to the present disclosure can be easily performed. Therefore, the test reagent of the present disclosure is preferably used in the first test method according to the present disclosure.

It can also be said that the present disclosure relates to use of a reagent for measuring radical scavenging ability in order to test the likelihood of physical frailty, for example.

The reagents may be mixed and provided, or some or all of the reagents may be provided separately. In the latter case, the test reagent according to the present disclosure can also be referred to as a test kit.

The test reagent according to the present disclosure may contain other constituents, for example. Examples of the other constituents include a pretreatment reagent for biological samples, instructions for use, and the like.

The test reagent according to the present disclosure may contain a reagent for measuring antioxidant, instead of the reagent for measuring radical scavenging ability. The reagent for measuring antioxidant can be appropriately determined depending on, for example, the type of the antioxidant. When the antioxidant is a protein, the test reagent may be a reagent for measuring antioxidant protein, or a reagent for measuring mRNA that encodes antioxidant protein, for example. It is possible to use an antibody against antioxidant or the like as the reagent for measuring antioxidant protein, for example. When the antioxidant is a nucleic acid, examples of the reagent for measuring mRNA that encodes the antioxidant protein include reverse transcriptase, dNTPs, polymerases, and primers. As a reagent for measuring the amount of antioxidant, for example, in the ORAC method, a high-purity Trolox standard product may be used as a standard.

### Diagnostic Method and Diagnostic Reagent for Physical Frailty

In another aspect, the present invention discloses a diagnostic method and a diagnostic reagent for likelihood of physical frailty. The diagnostic method for physical frailty according to the present disclosure includes measuring radical scavenging ability in a biological sample of a subject. The diagnostic reagent for physical frailty according to the present disclosure contains a reagent for measuring radical scavenging ability. Regarding the diagnostic method and diagnostic reagent for physical frailty, reference can be made to the descriptions as to the test method and the test reagent of the present disclosure.

### First Screening Method

In another aspect, the present invention discloses a method for screening therapeutic candidate substances for physical frailty. As described above, the method for screening therapeutic candidate substances for physical frailty according to the present disclosure includes selecting, from test substances, an activating substance that reduces radical scavenging ability as a therapeutic candidate substance for physical frailty. In the present disclosure, the target of therapeutic candidate substances for physical frailty is the radical scavenging ability, and there is no particular limitation on other steps or conditions. Regarding the first screening method of the present disclosure, reference can be made to the descriptions as to the physical frailty marker, the first test method, and the test reagent of the present disclosure.

Examples of the test substance include low-molecular-weight compounds, peptides, proteins, and nucleic acids. Examples of the low-molecular-weight compound include libraries of known low-molecular-weight compounds. Examples of the peptide include linear, branched, or cyclic peptides, and amino acids that constitute a peptide are natural amino acids, modified amino acids, artificial amino acids, or a combination thereof. The protein may be either a native protein or an artificial protein. Examples of the protein include antibodies, growth factors, proliferators, and variants thereof.

The first selecting includes, for example, causing each of the test substances to be also present in a radical generation system, and measuring radical scavenging ability; and choosing the test substance as the therapeutic candidate substance when the radical scavenging ability obtained in the causing of each of the test substances to be also present in a radical generation system, and measuring radical scavenging ability is lower than that in a control in which the test substance is not present. Regarding measuring the radical scavenging ability in the causing of each of the test substances to be also present in a radical generation system and measuring radical scavenging ability, reference can be made to the description as to the method for measuring radical scavenging ability. The radical generation system includes, for example, cells from a physical frailty patient.

### Detection Method

In another aspect, the present invention discloses a detection method for radical scavenging ability that can be used to detect likelihood of physical frailty in a subject who has a suspected case of physical frailty. The first detection method according to the present disclosure includes detecting radical scavenging ability in a biological sample of the subject using a reagent for measuring radical scavenging ability. The detection method according to the present disclosure includes detecting radical scavenging ability in a biological sample of the subject, and there is no particular limitation on other steps or conditions. Regarding the first detection method of the present disclosure, reference can be made to the descriptions as to the physical frailty marker, the first test method, and the test reagent.

The subject who has a suspected case of physical frailty may be, for example, a person who is subjectively suspected of having physical frailty, or a person who has been determined to have a suspected case of physical frailty, or possibly have physical frailty, as a result of a medical examination by a doctor or the like. Examples of the persons who are subjectively suspected of having physical frailty include persons who have some subjective symptoms and persons who wish to undergo preventive examinations.

### Mental Frailty Marker Set

In another aspect, the present invention discloses a marker set (referred to as “markers” hereinafter) that is an indicator of mental frailty. The mental frailty markers according to the present disclosure are radical scavenging ability and superoxide dismutase (SOD). The mental frailty markers according to the present disclosure are radical scavenging ability and SOD, and there is no particular limitation on other constituents or conditions.

As a result of intensive studies, the inventors of the present invention found that the SOD activity in a living organism, in particular the SOD activity in saliva, correlates with the development of mild cognitive impairment (MCI). As described above, the inventors of the present invention found that the radical scavenging ability in a living organism correlates with the development of physical frailty. It is believed that the mental frailty generally results from the development of MCI in addition to physical frailty. Therefore, according to the present disclosure, the likelihood (morbidity risk) of subjects having mental frailty and the like can be tested by measuring the SOD activity and the radical scavenging ability. Also, in the present disclosure, because the SOD activity and the radical scavenging ability serve as targets with respect to mental frailty, therapeutic candidate substances for mental frailty can be obtained through screening by using the targets.

Information registered in existing databases can be referenced for SOD derived from various animals. Specific examples of a human-derived SOD include cDNA, e.g., a polynucleotide consisting of the following base sequence (Sequence ID No. 1) registered as NCBI accession number NM_000454.5, and a protein, e.g., the following amino acid sequence (Sequence ID No. 2) registered as NCBI accession number NP_000445.1. The base sequence of Sequence ID No. 1 is a sequence that encodes the amino acid sequence of Sequence ID No. 2.

Human SOD cDNA
(SEQ ID NO: 1)
5′-GCGTCGTAGTCTCCTGCAGCGTCTGGGGTTTCCGTTGCAGTCCTCG
GAACCAGGACCTCGGCGTGGCCTAGCGAGTTATGGCGACGAAGGCCGTG
TGCGTGCTGAAGGGCGACGGCCCAGTGCAGGGCATCATCAATTTCGAGC
AGAAGGAAAGTAATGGACCAGTGAAGGTGTGGGGAAGCATTAAAGGACT
GACTGAAGGCCTGCATGGATTCCATGTTCATGAGTTTGGAGATAATACA
GCAGGCTGTACCAGTGCAGGTCCTCACTTTAATCCTCTATCCAGAAAAC
ACGGTGGGCCAAAGGATGAAGAGAGGCATGTTGGAGACTTGGGCAATGT
GACTGCTGACAAAGATGGTGTGGCCGATGTGTCTATTGAAGATTCTGTG
ATCTCACTCTCAGGAGACCATTGCATCATTGGCCGCACACTGGTGGTCC
ATGAAAAAGCAGATGACTTGGGCAAAGGTGGAAATGAAGAAAGTACAAA
GACAGGAAACGCTGGAAGTCGTTTGGCTTGTGGTGTAATTGGGATCGCC
CAATAAACATTCCCTTGGATGTAGTCTGAGGCCCCTTAACTCATCTGTT
ATCCTGCTAGCTGTAGAAATGTATCCTGATAAACATTAAACACTGTAAT
CTTAAAAGTGTAATTGTGTGACTTTTTCAGAGTTGCTTTAAAGTACCTG
TAGTGAGAAACTGATTTATGATCACTTGGAAGATTTGTATAGTTTTATA
AAACTCAGTTAAAATGTCTGTTTCAATGACCTGTATTTTGCCAGACTTA
AATCACAGATGGGTATTAAACTTGTCAGAATTTCTTTGTCATTCAAGCC
TGTGAATAAAAACCCTGTATGGCACTTATTATGAGGCTATTAAAAGAAT
CCAAATTCAAACTAAA-3′
Human SOD
(SEQ ID NO: 2)
MATKAVCVLKGDGPVQGIINFEQKESNGPVKVWGSIKGLTEGLHGFHVH
EFGDNTAGCTSAGPHFNPLSRKHGGPKDEERHVGDLGNVTADKDGVADV
SIEDSVISLSGDHCIIGRTLVVHEKADDLGKGGNEESTKTGNAGSRLAC
GVIGIAQ

The SOD activity combined with the radical scavenging ability can be used as a mental frailty marker, and can be particularly suitably used as a mental frailty marker caused by vascular diseases due to oxidative stress. Examples of the vascular disease due to stress include cardiovascular disease, respiratory disease, cranial nervous system disease, gastrointestinal disease, hematological disease, endocrine disease, urological disease, skin disease, supporting tissue disease, ophthalmic disease, tumor, iatrogenic disease, environmental pollution disease, and dental disease. The cranial nerve disease may include cerebral infarction, cerebral edema, and cerebral hemorrhage, which are cerebrovascular diseases; and dementia and depression, autonomic neuropathy (Reilly phenomena), which are neurodegenerative diseases. The cardiovascular disease may include myocardial infarction, arrhythmia, arteriosclerosis, vasospasm, and ischemia-reperfusion injury.

As will be described below, the mental frailty marker can also be used as a marker for diagnosing or detecting mental frailty, a marker for predicting the prognosis of mental frailty, or a marker for predicting or determining therapeutic effects on mental frailty.

### Mental Frailty Test Method

In another aspect, the present invention discloses a method for testing likelihood of mental frailty. The test method for mental frailty of the present disclosure includes measuring radical scavenging ability and SOD activity in a biological sample of a subject, as described above. The second test method of the present disclosure includes measuring, as mental frailty markers, the radical scavenging ability value and the SOD activity in the biological sample of the subject, and there is no particular limitation on other steps or conditions. Regarding the second test method of the present disclosure, reference can be made to the descriptions as to the physical frailty marker, the mental frailty marker and the first test method of the present disclosure.

With the second test method of the present disclosure, it is possible to perform testing for the likelihood of mental frailty, for example. The “testing for mental frailty” refers to detection of mental frailty, determination of mental frailty, screening for mental frailty, determination of a mental frailty preventive effect, determination of a mental frailty therapeutic effect, determination of a mental frailty patient who responds to a therapeutic agent, determination of a therapeutic agent that takes an effect on an individual mental frailty patient, a test method for diagnosing mental frailty, or a test for treating mental frailty. The “determination of mental frailty” may be, for example, determining whether mental frailty occurs; testing, detection or diagnosis of mental frailty; determination, testing, detection, or diagnosis of the likelihood (risk) of having mental frailty; prediction of prognosis after treatment of mental frailty; or determination of the therapeutic effect of a therapeutic agent for mental frailty.

Further, with the mental frailty marker, for example, mental frailty of a subject can be tested by the radical scavenging ability and the SOD activity in saliva. Therefore, the biological sample is preferably saliva because it is possible to reduce the burden on patients and doctors, for example.

Examples of the SOD activity to be measured include the superoxide (O₂·⁻) scavenging ability (scavenging activity) of SOD, for example. The SOD activity may be measured by measuring the O₂·⁻ scavenging ability of SOD for the biological sample, or it may be measured by measuring the O₂·⁻ scavenging ability of SOD for SOD obtained from the biological sample after extraction, rough purification, or purification, for example. There is no particular limitation on the method for measuring the O₂·⁻ scavenging ability of SOD, and a known method can be adopted. Specific examples of the method for measuring the O₂·⁻ scavenging ability of SOD include the Electron Spin Resonance (ESR) spin-trapping method using xanthine and xanthine oxidase as O₂·⁻ generating agents and a spin-trapping agent; spectrophotometry using tetrazolium salts (color formers) such as nitro blue tetrazolium (NBT) and 2,3,5-triphenyltetrazolium chloride (XTT); a chemiluminescent method using a chemiluminescent probe such as Cypridina luciferin analog (MCLA) and lucigenin; a method utilizing reduction of cytochrome C; a method utilizing reduction of tetranitromethane (TNM); a method utilizing oxidation (chain reaction) of epinephrine (adrenaline); a method utilizing oxidation (chain reaction) of lactate dehydrogenase-amyotrophic lateral sclerosis (NADH); and a measurement method through the formation of a lactoperoxidase/superoxide complex, furthermore the ESR spin-trapping method is preferable because it is possible to suppress the effects of other reactive oxygen species such as hydrogen peroxide, hydroxyl radicals, and singlet oxygen and to specifically detect O₂·⁻, and the ESR spin-trapping method has good sensitivity. The SOD activity may be measured qualitatively or quantitatively. When the SOD activity is measured quantitatively, the SOD activity can also be referred to as an SOD activity value.

The second test method of the present disclosure further includes, for example, testing the likelihood of the subject having mental frailty by comparing the radical scavenging ability value in the biological sample of the subject (also referred to as a “test biological sample” hereinafter) with a first threshold and by comparing the SOD activity value in the test biological sample with a second threshold.

The first threshold is not particularly limited, and may be, for example, a threshold calculated from radical scavenging ability values in biological samples of healthy subjects, radical scavenging ability values in biological samples of physical frailty patients, or the radical scavenging ability values in the biological samples of the healthy subjects and the physical frailty patients. In the case of prognostic assessment, the first threshold may be the radical scavenging ability value obtained before or after (e.g., immediately after) treating the same subject, for example.

The first threshold can be obtained using biological samples (also referred to as “reference biological samples” hereinafter) isolated from healthy subjects and/or physical frailty patients or mental frailty patients as described above, for example. Specifically, reference biological samples are isolated from multiple healthy subjects and multiple physical frailty patients or mental frailty patients, the radical scavenging ability thereof is measured, and then the first threshold may be calculated statistically, for example based on the obtained radical scavenging ability. In this case, as the first threshold, for example, clinical diagnosis values such as a diagnosis threshold (cut-off value), a treatment threshold, and a preventive medicine threshold, and the like can be used. Also, in the case of prognostic assessment, a reference biological sample isolated from the same subject after treatment may be used, for example. The first threshold may be measured at the same time as the measurement for the test biological sample from the subject or may be measured in advance, for example. The latter case is preferable because there is no need to obtain the first threshold each time a test biological sample from the subject is measured, for example. It is preferable that the test biological sample from the subject and the reference biological sample are collected under the same conditions, and the radical scavenging abilities therein are measured under the same conditions, for example.

The second threshold is not particularly limited, and may be, for example, a threshold calculated from SOD activity values in biological samples of healthy subjects, SOD activity values in biological samples of MCI patients, SOD activity values in biological samples of mental frailty patients, or the SOD activity values in the biological samples of the healthy subjects and the MCI patients or the mental frailty patients. In the case of prognostic assessment, the second threshold may be the SOD activity value obtained before or after (immediately after) treating the same subject, for example.

The second threshold can be obtained using biological samples (also referred to as “reference biological samples” hereinafter) isolated from healthy subjects and MCI patients or mental frailty patients as described above, for example. Specifically, reference biological samples are isolated from multiple healthy subjects and multiple MCI patients or mental frailty patients, the SOD activity thereof is measured, and then the second threshold may be calculated statistically, for example, based on the obtained SOD activity. In this case, as the second threshold, for example, clinical diagnosis values such as a diagnosis threshold (cut-off value), a treatment threshold, and a preventive medicine threshold, and the like can be used. Also, in the case of prognostic assessment, a reference biological sample isolated from the same subject after treatment may be used, for example. The second threshold may be measured at the same time as the measurement for the test biological sample from the subject or may be measured in advance, for example. The latter case is preferable because there is no need to obtain the second threshold each time a test biological sample from the subject is measured, for example. It is preferable that the test biological sample from the subject and the reference biological sample are collected under the same conditions, and the SOD activities therein are measured under the same conditions, for example.

There is no particular limitation on the method for assessing the likelihood of a subject having mental frailty in the second testing, and the assessment method can be determined as appropriate according to the type of the threshold. As a specific example, in the testing, the biological sample of the subject may be assessed based on whether or not it satisfies the combination of the following conditions (1) to (4). The following conditions (1) and (2) are criteria for assessing whether or not the subject has physical frailty. The following conditions (3) and (4) are criteria for assessing whether or not the subject has MCI. Therefore, in the following examples, in the second testing, it is possible to assess whether or not the subject has mental frailty by assessing the combination of the following condition (1) or (2) and the following condition (3) or (4).

### Condition (1)

When the radical scavenging ability value in the test biological sample from the subject is (significantly) higher than the radical scavenging ability values in the reference biological samples of the healthy subjects, when the radical scavenging ability value in the test biological sample from the subject is the same as the radical scavenging ability values in the reference biological samples of the physical frailty patients (when there is no significant difference), when the radical scavenging ability value in the test biological sample from the subject is (significantly) higher than the radical scavenging ability values in the reference biological samples of the physical frailty patients, and/or when the radical scavenging ability value in the test biological sample from the subject is higher than a first threshold calculated from the radical scavenging ability values in the biological samples of the healthy subjects and the physical frailty patients or the mental frailty patients;

### Condition (2)

When the radical scavenging ability value in the test biological sample from the subject is (significantly) lower than the radical scavenging ability values in the reference biological samples of the healthy subjects, when the radical scavenging ability value in the test biological sample from the subject is the same as the radical scavenging ability values in the reference biological samples of the healthy subjects (when there is no significant difference), when the radical scavenging ability value in the test biological sample from the subject is (significantly) lower than the radical scavenging ability values in the reference biological samples of the physical frailty patients, and/or when the radical scavenging ability value in the test biological sample from the subject is lower than the first threshold calculated from the radical scavenging ability values in the biological samples of the healthy subjects and the physical frailty patients or the mental frailty patients;

### Condition (3)

When the SOD activity value in the test biological sample from the subject is (significantly) higher than the SOD activity values in the reference biological samples of the healthy subjects, when the SOD activity value in the test biological sample from the subject is the same as the SOD activity values in the reference biological samples of the healthy subjects (when there is no significant difference), when the SOD activity value in the test biological sample from the subject is (significantly) higher than the SOD activity values in the reference biological samples of the MCI patients or the mental frailty patients, and/or when the SOD activity value in the test biological sample from the subject is higher than the second threshold calculated from the SOD activity values in the biological samples of the healthy subjects and the MCI patients or the mental frailty patients;

### Condition (4)

When the SOD activity value in the test biological sample from the subject is (significantly) lower than the SOD activity values in the reference biological samples of the healthy subjects, when the SOD activity value in the test biological sample from the subject is the same as the SOD activity values in the reference biological samples of the MCI patients or the mental frailty patients (when there is no significant difference), when the SOD activity value in the test biological sample from the subject is (significantly) lower than the SOD activity values in the reference biological samples of the MCI patients or the mental frailty patients, and/or when the SOD activity value in the test biological sample from the subject is lower than the second threshold calculated from the SOD activity values in the biological samples of the healthy subjects and the MCI patients or the mental frailty patients.

In the second test method, for example, when the biological sample of the subject satisfies the conditions (1) and (4) in the second testing, the subject can be assessed as likely to or highly likely (having high risk) to have mental frailty. In the second testing, when the biological sample of the subject satisfies the conditions (1) and (4), the subject may also be assessed as likely to or highly likely (having high risk) to have physical frailty and/or likely to or highly likely (having high risk) to have MCI.

In the second test method, for example, when the biological sample of the subject satisfies the conditions (1) and (3) in the second testing, the subject can be assessed as having no or little likelihood (risk) of having mental frailty. In the second testing, when the biological sample of the subject satisfies the conditions (1) and (3), the subject may also be assessed as highly likely to have physical frailty and/or having no or little likelihood (risk) of having MCI.

In the second test method, for example, when the biological sample of the subject satisfies the conditions (2) and (4) in the second testing, the subject can be assessed as having no or little likelihood (risk) of having mental frailty. In the second testing, when the biological sample of the subject satisfies the conditions (2) and (4), the subject may also be assessed as having no or little likelihood (risk) of having physical frailty and/or likely to or highly likely (having high risk) to have MCI.

In the second test method, for example, when the biological sample of the subject satisfies the conditions (2) and (3) in the second testing, the subject can be assessed as having no or little likelihood (risk) of having mental frailty. In the second testing, when the biological sample of the subject satisfies the conditions (2) and (3), the subject may also be assessed as having no or little likelihood (risk) of having physical frailty and/or having no or little likelihood (risk) of having MCI.

In the second testing, the level of mental frailty may be assessed by comparing the radical scavenging ability value and the SOD activity value in the test biological sample from the subject with the radical scavenging ability values and the SOD activity values in the reference biological samples of the mental frailty patients at respective levels of mental frailty. Specifically, when the test biological sample from the subject has a radical scavenging ability value and an SOD activity value similar to those in the reference biological samples at a certain level of mental frailty (when there is no significant difference), for example, the subject can be assessed as likely to have mental frailty at that level.

When the prognostic state is assessed in the second testing, an assessment may be made in the same manner as the above-described manner or can be assessed using, as the first threshold and the second threshold, the radical scavenging ability value and the SOD activity value in a reference biological sample obtained after treating the same subject, for example. As specific examples, when the radical scavenging ability value in the test biological sample from the subject is the same as the first threshold and the SOD activity value in the test biological sample from the subject is the same as the second threshold (when there is no significant difference), and/or when the radical scavenging ability value in the test biological sample from the subject is (significantly) higher than the first threshold and the SOD activity value in the test biological sample from the subject is (significantly) lower than the second threshold, the subject can be assessed as likely to relapse or worsen after the treatment. Also, when the radical scavenging ability value in the test biological sample from the subject is (significantly) lower than the first threshold and/or the SOD activity value in the test biological sample from the subject is (significantly) higher than the second threshold, the subject can be assessed as having no or little likelihood of a relapse of mental frailty after the treatment.

In the second testing, biological samples may be collected from the same subject over time, and the radical scavenging ability values and the SOD activity values in the biological samples may be compared to each other, for example. As a result, in the second testing, when the radical scavenging ability value increases and the SOD activity value decreases over time, it can be determined that the likelihood of having mental frailty has increased, and, when the radical scavenging ability value decreases and/or the SOD activity value increases, it can be determined that the likelihood of having mental frailty has decreased or the patient has been cured, for example.

In the second test method according to the present disclosure, treatment may be administered to the subject based on the results of the second testing, for example. Specifically, the second test method according to the present disclosure may include administering a therapeutic agent for mental frailty to a subject who has received a test result indicating that the subject has mental frailty, for example. Conditions such as an administration form, an administration period, a dosage, and an administration interval of the therapeutic agent for mental frailty and the like can be set as appropriate according to the type of the therapeutic agent for mental frailty. The therapeutic agent for mental frailty may be a therapeutic candidate substance obtained through the screening method according to the present disclosure to be described below.

As described above, the second test method according to the present disclosure can be used as a method for diagnosing or detecting mental frailty, a method for predicting the prognosis of mental frailty, and a method for predicting or determining effects of mental frailty treatment, for example. Therefore, the second test method according to the present disclosure can also be used as a companion diagnostic method for selecting patients (responders) who respond to the therapeutic agent for mental frailty and adjusting the dosage of the therapeutic agent for mental frailty.

### Test Kit

In another aspect, the present invention discloses a test kit for testing the likelihood of mental frailty. As described above, the test kit according to the present disclosure contains a reagent for measuring radical scavenging ability and a reagent for measuring SOD activity. The test kit according to the present disclosure contains a reagent for measuring radical scavenging ability and a reagent for measuring SOD activity, and there is no particular limitation on other constituents or conditions. With the test kit according to the present invention, the test method according to the present invention can be easily performed. Regarding the test kit of the present disclosure, reference can be made to the descriptions as to the mental frailty marker and the second test method of the present disclosure.

The reagent for measuring radical scavenging ability can be appropriately determined according to, for example, a method for measuring the radical scavenging ability. Specifically, when the radical scavenging ability is measured by the ESR method such as the i-STrap method, examples of the reagent for measuring radical scavenging ability include a radical generating agent and a radical detecting agent. The radical generating agent may include a lipid radical generating agent, and the radical detecting agent may include a lipid radical detecting agent. Examples of the radical generating agent include a combination of tBuOOH and hemoglobin. Examples of the radical generating agent include tert-butyl hydroperoxide (tBuOOH) and hemoglobin. Examples of the radical detecting agent include 2-diphenylphosphinoyl-2-methyl-3,4-dihydro-2H-pyrrole N-oxide (DPhPMPO), N-tert-Buthyl-α-Phenylnitrone (PBN), α-(4-Pyridyl-1-Oxide)-N-tert-Butylnitrone (4-POBN), 3,3,5,5-Tetramethyl-1-Pyrroline-N-Oxide (M4PO), 3,5-Dibromo-4-Nitrosobenzenesulfonic Acid, Sodium Salt (DBNBS), 3,5-Dibromo-4-Nitrosobenzenesulfonic Acid-d2, and Sodium Salt (DBNBS-d2). The reagent for measuring SOD activity can be determined as appropriate according to the method for measuring the SOD activity, for example. When the SOD activity is to be measured using the Electron Spin Resonance (ESR) method, specific examples of the reagent for measuring SOD activity include a superoxide (O₂·⁻) generating agent and a spin-trapping agent. Examples of the O₂·⁻ generating agent include a combination of xanthine and xanthine oxidase. Examples of the spin-trapping agent include 5,5-dimethyl-1-pyrroline-N-oxide (DMPO), 5-(diethoxyphophoryl)-5-methyl-1-pyrroline-N-oxide (DEPMPO), and 5-(2,2-dimethyl-1,3-propoxycyclophosphoryl)-5-methyl-1-pyrroline N-oxide (CYPMPO).

As described above, with the test kit according to the present disclosure, the second test method according to the present disclosure can be easily performed. Therefore, the test kit of the present disclosure is preferably used in the second test method according to the present disclosure.

It can also be said that the present disclosure relates to the use of a reagent for measuring radical scavenging ability and a reagent for measuring SOD activity for testing the likelihood of mental frailty, for example.

In the present disclosure, reagent kits may be mixed and provided, or some or all of the reagent kits may be provided separately. When the reagent kits are mixed and provided, the test kit according to the present disclosure can also be referred to as, for example, a test reagent for mental frailty.

The test kit according to the present disclosure may contain other constituents, for example. Examples of the other constituents include a pretreatment reagent for biological samples, instructions for use, and the like.

The test kit according to the present disclosure may contain a reagent for measuring antioxidant, instead of the reagent for measuring radical scavenging ability. The reagent for measuring antioxidant may be a reagent for measuring antioxidant protein, or a reagent for measuring mRNA that encodes antioxidant protein, for example. It is possible to use an antibody against an antioxidant or the like as the reagent for measuring antioxidant protein, for example. Examples of the reagent for measuring mRNA that encodes the antioxidant protein include reverse transcriptase, dNTPs, polymerases, and primers. The test kit according to the present disclosure may contain a reagent for measuring SOD, instead of the reagent for measuring the SOD activity. The reagent for measuring SOD may be a reagent for measuring SOD protein, or a reagent for measuring mRNA that encodes SOD protein, for example. It is possible to use an antibody against SOD or the like as the reagent for measuring SOD protein, for example. When the antioxidant is a nucleic acid, examples of the reagent for measuring mRNA that encodes the SOD protein include reverse transcriptase, dNTPs, polymerases, and primers.

### Diagnostic Method and Diagnostic Reagent for Mental Frailty

In another aspect, the present invention discloses a diagnostic method and a diagnostic reagent for likelihood of mental frailty. The diagnostic method for mental frailty according to the present disclosure includes measuring radical scavenging ability value and SOD activity in a biological sample of a subject. The diagnostic reagent for mental frailty according to the present disclosure contains a reagent for measuring radical scavenging ability and a reagent for measuring SOD activity. Regarding the diagnostic method and diagnostic reagent for mental frailty, reference can be made to the descriptions as to the first test method, the second test method, the test reagent, and the test kit of the present disclosure.

### Second Screening Method

In another aspect, the present invention discloses a method for screening therapeutic candidate substances for mental frailty. As described above, the method for screening therapeutic candidate substances for mental frailty according to the present disclosure includes selecting from test substances, an activating substance that reduces radical scavenging ability and increases SOD activity as a therapeutic candidate substance for mental frailty. In the present disclosure, the target of therapeutic candidate substances for mental frailty is the radical scavenging ability and the SOD activity, and there is no particular limitation on other steps or conditions. Regarding the second screening method of the present disclosure, reference can be made to the descriptions as to the mental frailty marker, the second test method, and the test kit of the present disclosure.

Examples of the test substance include low-molecular-weight compounds, peptides, proteins, and nucleic acids. Examples of the low-molecular-weight compounds include libraries of known low-molecular-weight compounds. Examples of the peptides include linear, branched, or cyclic peptides, and amino acids that constitute a peptide are natural amino acids, modified amino acids, artificial amino acids, or a combination thereof. The protein may be either a native protein or an artificial protein. Examples of the protein include antibodies, growth factors, proliferators, and variants thereof. Examples of the nucleic acids include substances that suppress the expression of SOD, and specific examples thereof include substances that suppress transcription of mRNA from the SOD gene, substances that cleave the transcribed mRNA, and substances that suppress protein translation from mRNA. Examples of the nuclei acids include RNA interference agents such as siRNA, antisense, and ribozymes.

The second selecting includes, for example, causing each of the test substances to be also present in a radical generation system, and measuring radical scavenging ability (first measuring); and causing each of the test substances to be also present in a system containing superoxide and SOD, and measuring SOD activity (second measuring). The second selecting includes, for example, choosing the test substance as the therapeutic candidate substance when the radical scavenging ability obtained in the first measuring is lower than that in a control system in which the test substance is not present and when the SOD activity obtained in the second measuring is higher than that in a control system in which the test substance is not present. In the first measuring, regarding the measurement of the radical scavenging ability, for example, reference can be made to the description as to the method for measuring radical scavenging ability. In the second measuring, regarding the measurement of the SOD activity, for example, reference can be made to the description as to the measurement of the SOD activity. The radical generation system and the mixture system containing superoxide and SOD can be prepared, for example, using the test kit. The radical generation system includes, for example, cells from a physical frailty patient.

### Detection Method

In another aspect, the present invention discloses a detection method for radical scavenging ability and SOD activity that can be used to detect the likelihood of mental frailty in a subject who has a suspected case of mental frailty. The detection method according to the present disclosure is a detection method for radical scavenging ability and SOD activity in a subject who has a suspected case of mental frailty, including detecting radical scavenging ability in a biological sample of the subject using a reagent for measuring radical scavenging ability (first detecting); and detecting SOD activity in the biological sample of the subject using a reagent for measuring SOD activity (second detecting). The detection method according to the present disclosure includes detecting radical scavenging ability and SOD activity in a biological sample of the subject, and there is no particular limitation on other steps or conditions. Regarding the second detection method of the present disclosure, reference can be made, for example, to the descriptions as to the mental frailty marker, the second test method, the test reagent, and the test kit of the present disclosure.

The subject who has a suspected case of mental frailty may be, for example, a person who is subjectively suspected of having mental frailty, or a person who has been determined to have a suspected case of mental frailty, or possibly has mental frailty, as a result of a medical examination by a doctor or the like. Examples of the persons who are subjectively suspected of having mental frailty include persons who have some subjective symptoms and persons who wish to undergo preventive examinations.

### EXAMPLES

The following describes examples of the present invention. However, the present invention is not limited to the following examples. Commercially available reagents were used based on protocols thereof, unless otherwise stated.

### Example 1

The present invention was used to analyze the radical scavenging ability of saliva.

### (1) Subject

Subjects were divided into three groups. Specifically, the subjects were divided into (1) a group aged under 50 years (n=18), (2) a group aged 50 years or older and under 70 years (n=32), and (3) a group aged 70 years or older (n=32). It was examined whether the radical scavenging ability correlates with the physical frailty.

### (2) Collection of Saliva

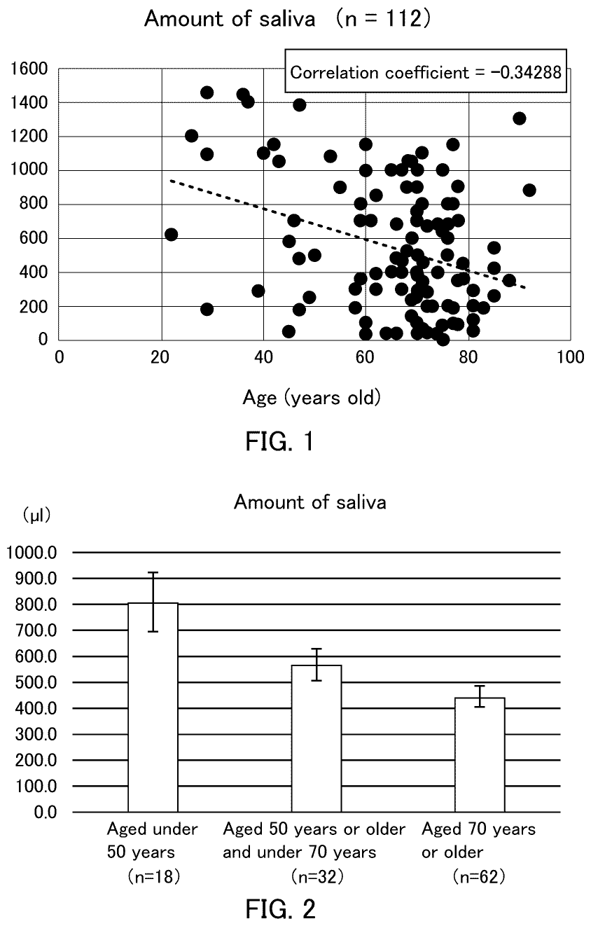

Saliva was collected using a commercially available saliva collection tube (SALIKIDS® manufactured by Sarstedt K.K.), as follows. First, each subject was allowed to introduce a cotton roll attached to SALIKIDS® into the oral cavity for 5 minutes so that the cotton roll sufficiently absorbed the saliva of the subject. Then, the cotton roll impregnated with saliva was inserted into a suspending insert of SALIKIDS®, a cap was closed, and then centrifugation was performed under the conditions at 3000×g for 3 minutes. After separation was performed, the supernatant fraction in the tube was used as a saliva sample of each subject and stored at −40° C. Note that when saliva was collected, each subject was prohibited from eating, drinking, taking drugs orally, brushing teeth, and having Professional Mechanical Tooth Cleaning (PMTC) for 1 hour before the collection thereof. The amount of saliva was measured for the collected saliva. The results are shown in FIGS. 1 and 2.

### (3) Amount of Saliva

FIGS. 1 and 2 show the amounts of saliva of the subjects. FIG. 1 shows the results of the amounts of saliva of the subjects. In FIG. 1, the vertical axis indicates the amount of saliva of the subject, and the horizontal axis indicates the age of the subject. FIG. 2 is a graph showing the average of the amounts of saliva of the subjects in each age group. In FIG. 2, the vertical axis indicates the amount of saliva in each age group, and the horizontal axis indicates the age group of the subject. As shown in FIG. 1, the correlation coefficient between the age of the subject and the amount of saliva was −0.34288, and it was found that there was a negative correlation between the amount of saliva and the age. As shown in FIG. 2, the average of the amounts of saliva decreased in the group aged 50 years or older and under 70 years compared to the group aged under 50 years. The average of the amounts of saliva was further decreased in the group aged 70 years or older compared to the group aged under 50 years and the group aged 50 years or older and under 70 years.

### (4) Grip Strength

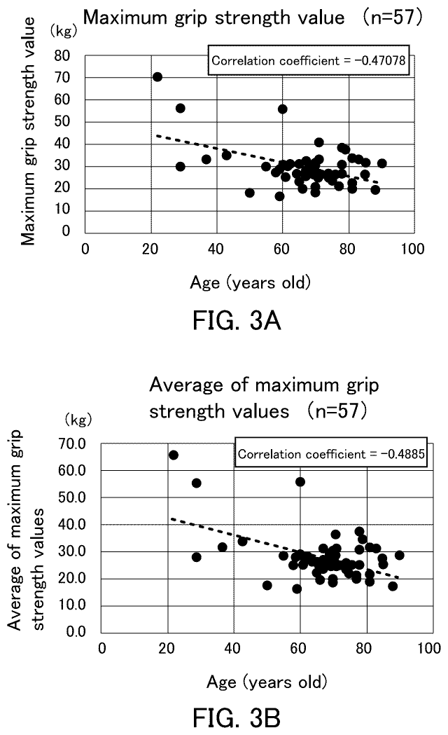

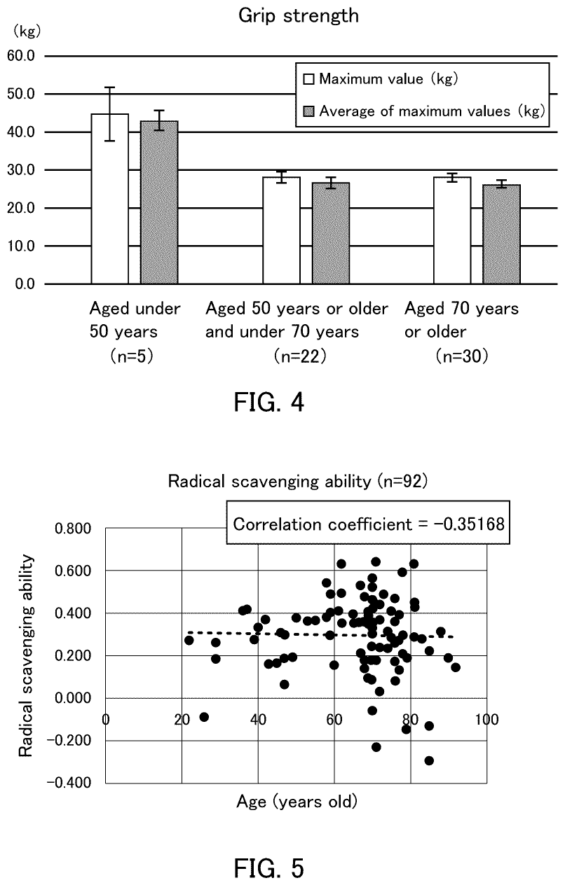

The physical frailty shows a decrease in physical performance. The physical performance of the subject was measured by examining whether the radical scavenging ability correlates with the physical frailty. Specifically, the maximum grip strength values of the subjects were measured. For the measurement of grip strength, a grip dynamometer was used, and measurement was made twice (once when it was difficult to measure twice) on the left and right hands in the standing position. The results are shown in FIGS. 3A, 3B, and 4.

FIGS. 3A, 3B, and 4 are graphs showing the grip strength of the subjects. FIG. 3A shows the results of the maximum grip strength values of the subjects, and FIG. 3B shows the results of the average of the maximum grip strength values of the subjects. In FIGS. 3A and 3B, the vertical axis indicates the grip strength of the subject, and the horizontal axis indicates the age of the subject. FIG. 4 is a graph showing the maximum grip strength values of the subjects in each age group and the average of the maximum grip strength values of the subjects in each age group. In FIG. 4, the vertical axis indicates the grip strength of each age group, and the horizontal axis indicates the age group of the subject. As shown in FIGS. 3A and 3B, as to the correlation coefficient, the maximum value of grip strength was −0.47048, the average of the maximum values of the right hand was −0.47936, the average of the maximum values of the left hand was −0.48541, and the average of the maximum values of both hands was −0.4885. From these results, it was found that there is a negative correlation between the grip strength and the age of the subject. Next, as shown in FIG. 4, the maximum grip strength value in each age group and the average of the maximum grip strength values in each age group decreased in the group aged 50 years or older and under 70 years compared to the group aged under 50 years. The maximum grip strength value in each age group and the average of the maximum grip strength values in each age group further decreased in the group aged 70 years or older compared to the group aged under 50 years, and the group aged 50 years or older and under 70 years. As a preliminary stage to sarcopenia, physical frailty is first caused by impaired physical function. The sarcopenia is caused by a further loss of muscle mass or the like in the state of physical frailty. Therefore, in the present case, it was presumed that the group aged 50 years or older and under 70 years was a group with physical frailty, and the group aged 70 years or older was a group with sarcopenia caused by loss of muscle mass.

### (5) Measurement of Radical Scavenging Ability of Saliva

The radical scavenging ability of each saliva sample was measured by the i-STrap (Dojindo Laboratories), which is an electron spin resonance (ESR) spin-trapping method using 2-diphenylphosphinoyl-2-methyl-3,4-dihydro-2H-pyrrole N-oxide (DPhPMPO) as a spin-trapping agent. The i-Strap was carried out according to Reference 1 below. Specifically, a DPhPMPO solution (final concentration: 10 mmol/1), 80 μl of saline, and 20 μl of hemoglobin were mixed to obtain a mixed solution. 100 μl of saliva collected in Example 1 (2) was added to the mixed solution, and then the obtained reactant was mixed using a stirrer (VORTEX-GENIE2 Mixer, manufactured by M&S Instruments Inc.). After the mixing, 20 μl of tBuOOH (final concentration: 10 mmol/l) was added, membrane lipid radical tert-butyl, tert-butyloxyl, and tert-butylperoxyl radical were generated by the Fenton reaction and incubated at room temperature (about 25° C., hereinafter the same applies) for 30 minutes. Then, 1 ml of a chloroform/methanol solution (Wako Pure Chemical Industries, Ltd.) mixed at a volume ratio of 2:1 was added, and the obtained mixed solution was stirred at room temperature for 10 minutes using the stirrer. Then the spin adduct was extracted from the mixed solution after stirring using a chloroform/methanol organic solvent. The samples obtained in the extraction (160 μl) were brought to room temperature, introduced into quartz flat cells (Cat. No: RST-LC09F, Flashpoint), and then X-band ESR spectroscopy was used to measure the radical scavenging ability of the saliva sample. The ESR measurement conditions are described below.
Reference 1: Sun, Lue, et al. “Dose-dependent decrease in antioxidant capacity of whole blood after irradiation: A novel potential marker for biodosimetry.” Scientific reports 8.1 (2018): 1-8.

Lipid radicals (tert-butyl, tert-butyloxyl, and tert-butylperoxyl radicals), which have not been scavenged by the radical scavenging ability in saliva, react with DPhPMPO added to the reaction system. After the reaction, DPhPMPO-tert-butyl, DPhPMPO-tert-butyloxyl, and DPhPMPO-tert-butylperoxyl, which are stable radicals detectable by ESR, are generated.

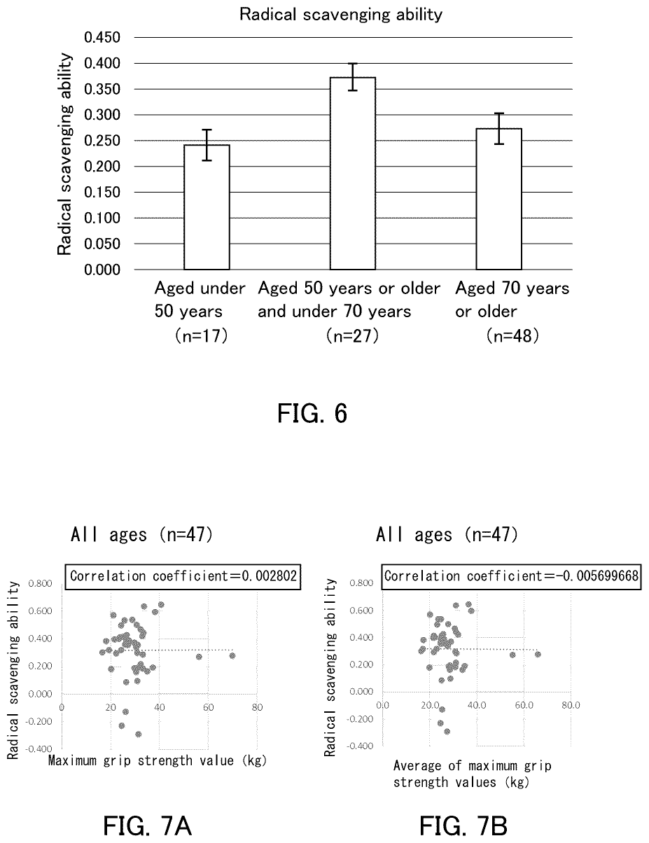

In the ESR measurement, an electron spin resonance device (Cat. No: JES-TE200, manufactured by JEOL Ltd.) was connected to WIN-RAD ESR data analyzer (Radical Research, Tokyo, Japan), and the following measurement conditions were used. The hyperfine coupling constant was calculated using the resonance frequency measured by using a microwave frequency counter and the resonance electric field was measured using an electric field measuring device ES-FC5 (JEOL Ltd.). The detected spin adducts were quantified from the ESR spectra of manganese oxide standards. The actual measured signal intensity was expressed by a relative height normalized to the signal intensity of the ESR spectra of the manganese oxide standards, thereby obtaining an i-STrap value. The radical scavenging ability was calculated by setting an i-STrap value in a reaction system to which a saliva sample is not added to 1 (reference value) and calculating the difference (1-R) between the reference value and an i-STrap value (R) in a reaction system to which a saliva sample is added. The average of radical scavenging ability values was calculated for each group. The results are shown in FIGS. 5 and 6.

### ESR Measurement Conditions

### Device:

Electron Spin Resonance (ESR) device (JES-TE200, X-band spectrometer, manufactured by JEOL Ltd.)

### Measurement Conditions:

Microwave frequency: 9.422 GHz
Microwave power: 2.00 mW
Sweep time: 4 minutes
Sweep width: 0.3 mT
Magnetic field center: 332.0 mT
Time constant: 0.3 seconds

FIGS. 5 and 6 are graphs showing the results of ESR in saliva samples. FIG. 5 shows the results of the radical scavenging ability of the subjects. In FIG. 5, the vertical axis indicates the radical scavenging ability of the subject, and the horizontal axis indicates the age of the subject. FIG. 6 shows the results of the average of radical scavenging ability values of the subjects in each age group. In FIG. 6, the vertical axis indicates the radical scavenging ability in each age group, and the horizontal axis indicates the age group of the subject. As shown in FIG. 5, the correlation coefficient between the age of the subject and the radical scavenging ability was −0.35168, and it was found that there was a negative correlation between the radical scavenging ability and the age. Further, as shown in FIG. 6, the average of the radical scavenging ability values increased in the group aged 50 years or older and under 70 years compared to the group aged under 50 years. The average of the radical scavenging ability values decreased in the group aged 70 years or older as compared to the group aged 50 years or older and under 70 years. Therefore, it was proven that the radical scavenging ability in the saliva was increased in the group with physical frailty.

### (6) Grip Strength and Antioxidant Ability

In order to examine whether the grip strength correlates with the radical scavenging ability in a group of physical frailty, the correlation between the grip strength and the radical scavenging ability was examined based on the results of physical performance and the radical scavenging ability of saliva of the subjects. Specifically, the correlation between the grip strength and the radical scavenging ability was examined using the grip strength values in Example 1 (4) and the radical scavenging ability values of saliva in Example 1 (5). The results are shown in FIGS. 7A and 7B.

FIGS. 7A and 7B are graphs showing the correlation between the grip strength and the radical scavenging ability of the subjects. FIG. 7A shows the results using the maximum grip strength values, and FIG. 7B shows the results using the average of the maximum grip strength values. In FIG. 7A, the vertical axis indicates the radical scavenging ability of the subject, and the horizontal axis indicates the maximum grip strength of the subject. In FIG. 7B, the vertical axis indicates the radical scavenging ability of the subject, and the horizontal axis indicates the average of the maximum grip strength values of the subjects. As shown in FIG. 7A, the correlation coefficient between the radical scavenging ability and the maximum grip strength values of the subjects was 0.002802, and it was found that there was no correlation between the radical scavenging ability and the maximum grip strength values when the age was not taken into consideration. As shown in FIG. 7B, the correlation coefficient between the radical scavenging ability and the average of the maximum grip strength values of the subjects was −0.005699668, and it was found that there was no correlation between the radical scavenging ability and the average of the maximum grip strength values when the age was not taken into consideration.

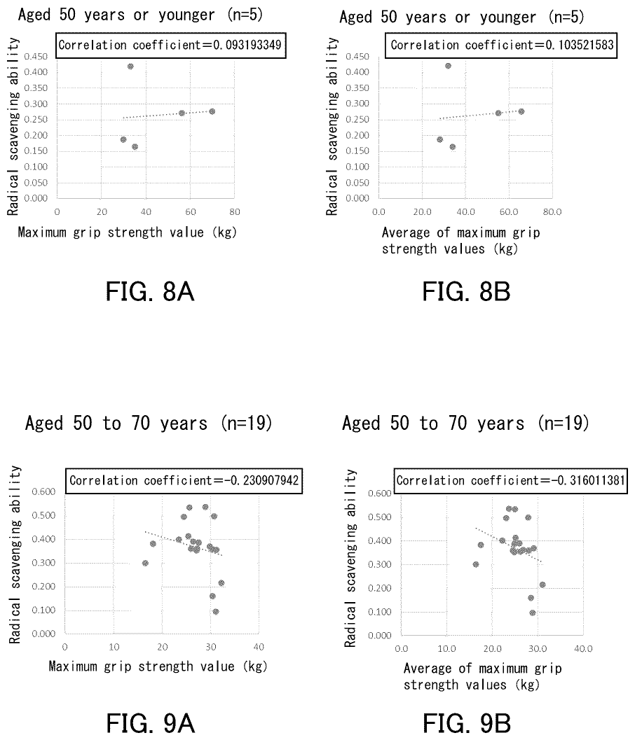

Next, in order to examine whether the correlation between the grip strength and the radical scavenging ability depends on age, the correlation between the grip strength and the radical scavenging ability was examined based on the results of physical performance and the radical scavenging ability of saliva of the subjects. Specifically, the correlation between the grip strength and the radical scavenging ability was examined using the grip strength values of the subjects aged 50 years or younger in Example 1 (4) and the radical scavenging ability values of saliva of the subjects aged 50 years or younger in Example 1 (5). The results are shown in FIG. 8.

FIGS. 8A and 8B are graphs showing the correlation between the grip strength and the radical scavenging ability of the subjects aged 50 years or younger. FIG. 8A shows the results using the maximum grip strength values, and FIG. 8B shows the results using the average of the maximum grip strength values. In FIG. 8A, the vertical axis indicates the radical scavenging ability of the subject, and the horizontal axis indicates the maximum grip strength value of the subject. In FIG. 8B, the vertical axis indicates the radical scavenging ability of the subject, and the horizontal axis indicates the average of the maximum grip strength values of the subject. As shown in FIG. 8A, the correlation coefficient between the radical scavenging ability and the maximum grip strength values of the subjects was 0.093193349, and it was found that there was no correlation between the radical scavenging ability and the maximum grip strength values in the subjects aged 50 years or younger. As shown in FIG. 8B, the correlation coefficient between the radical scavenging ability and the average of the maximum grip strength values of the subjects was 0.103521583, and it was found that there was no correlation between the radical scavenging ability and the average of the maximum grip strength values in the subjects aged 50 years or younger.

Next, in order to examine whether the correlation between the grip strength and the radical scavenging ability depends on age, the correlation between the grip strength and the radical scavenging ability was examined based on the results of physical performance and the radical scavenging ability of saliva of the subjects. Specifically, the correlation between the grip strength and the radical scavenging ability was examined using the grip strength values of the subjects aged 50 to 70 years in Example 1 (4) and the radical scavenging ability values of saliva of the subjects aged 50 to 70 years in Example 1 (5). The results are shown in FIG. 9.

FIGS. 9A and 9B are graphs showing the correlation between the grip strength and the radical scavenging ability of the subjects aged 50 to 70 years. FIG. 9A shows the results using the maximum grip strength values, and FIG. 9B shows the results using the average of the maximum grip strength values. In FIG. 9A, the vertical axis indicates the radical scavenging ability of the subject, and the horizontal axis indicates the maximum grip strength of the subject. In FIG. 9B, the vertical axis indicates the radical scavenging ability of the subject, and the horizontal axis indicates the average of the maximum grip strength values of the subject. As shown in FIG. 9A, the correlation coefficient between the radical scavenging ability and the maximum grip strength values of the subjects was −0.230907942, and it was found that there was a negative correlation between the radical scavenging ability and the maximum grip strength values in the subjects aged 50 to 70 years. As shown in FIG. 9B, the correlation coefficient between the radical scavenging ability and the average of the maximum grip strength values of the subjects was −0.316011381, and it was found that there was a negative correlation between the radical scavenging ability and the average of the maximum grip strength values in the subjects aged 50 to 70 years.

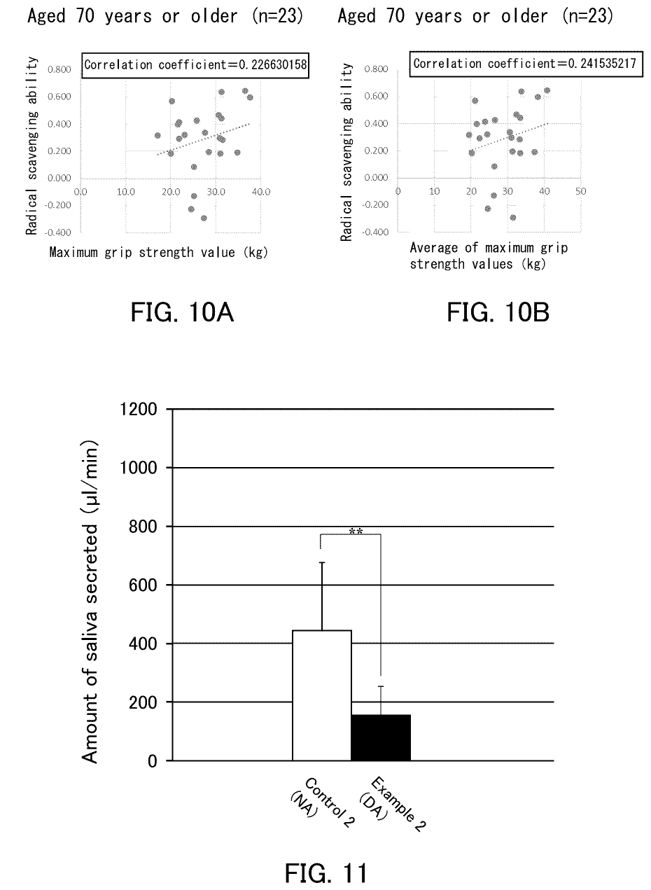

Next, in order to examine whether the correlation between the grip strength and the radical scavenging ability depends on age, the correlation between the grip strength and the radical scavenging ability was examined based on the results of physical performance and the radical scavenging ability of saliva of the subjects. Specifically, the correlation between the grip strength and the radical scavenging ability was examined using the grip strength values of the subjects aged 70 years or older in Example 1 (4) and the radical scavenging ability values of saliva of the subjects aged 70 years or older in Example 1 (5). The results are shown in FIG. 10.

FIGS. 10A and 10B are graphs showing the correlation between the grip strength and the radical scavenging ability of the subjects aged 70 years or older. FIG. 10A shows the results using the maximum grip strength values, and FIG. 10B shows the results using the average of the maximum grip strength values. In FIG. 10A, the vertical axis indicates the radical scavenging ability of the subject, and the horizontal axis indicates the maximum grip strength of the subject. In FIG. 10B, the vertical axis indicates the radical scavenging ability of the subject, and the horizontal axis indicates the average of the maximum grip strength values of the subject. As shown in FIG. 10A, the correlation coefficient between the radical scavenging ability and the maximum grip strength values of the subjects was 0.226630158, and it was found that there was a correlation between the radical scavenging ability and the maximum grip strength values in the subjects aged 70 years or older. As shown in FIG. 10B, the correlation coefficient between the radical scavenging ability and the average of the maximum grip strength values of the subjects was 0.241535217, and it was found that there was a correlation between the radical scavenging ability and the average of the maximum grip strength values in the subjects aged 70 years or older.

As described above, it was presumed that physical frailty occurs in the group aged 50 years or older and under 70 years. In the group aged 50 years or older and under 70 years, the grip strength and the radical scavenging ability are correlated. Therefore, it was found that it is possible to presume the level of the physical frailty by measuring the radical scavenging ability.

### Example 2

Down syndrome patients were used as mild cognitive impairment (MCI) models, and it was examined that the superoxide dismutase (SOD) activity decreased in saliva derived from the Down syndrome patients.

### (1) Subject

It is said that Down syndrome patients show MCI in an early stage. In view of this, whether the SOD activity correlates with MCI was examined using Down syndrome patients as MCI models.

A Down syndrome patient group (DA, Example 2, n=31, 22 males, nine females, average age 48.9±6.5 years), and a healthy subject group (NA, Control 2, n=24, seven males, 14 females, average age 47.1±4.9 years) were used as subjects. Each subject provided written informed consent. Also, when selecting subjects, persons who had used a corticosteroid or immunosuppressant agent for a long period, persons who had a history of use of antibiotics within the past three months, and persons who had been treated with an antifungal agent within the past six weeks were excluded.

### (2) Collection of Saliva

The saliva was collected in the same manner as in Example 1 (2). The amount of saliva was measured for the collected saliva. The results are shown in FIG. 11.

### (3) Amount of Saliva

FIG. 11 shows the amounts of saliva of the subjects. FIG. 11. is a graph showing the average of the amounts of saliva of the subjects in each group, and the vertical axis indicates the average of the amounts of saliva in each group, and the horizontal axis indicates the subject group. As shown in FIG. 11, the Down syndrome patient group (Example 2) exhibited a significant decrease in the amount of saliva as compared to the healthy subject group (Control 2). Note that the asterisk in FIG. 11. indicates a p-value, and ** means p<0.01. Also, statistical analysis was performed using the Student-Newman-Keuls method (the same applies to the following).

(4) Measurement of Superoxide (O₂·⁻) Scavenging Ability of Saliva

The superoxide (O₂·⁻) scavenging ability of each saliva sample was examined using the Electron Spin Resonance (ESR) spin-trapping method using 5,5-dimethyl-1-pyrroline-N-oxide (DMPO) as a spin-trapping agent. The O₂·⁻ production system was a xanthine/xanthine oxidase (XO) production system. Specifically, 20 μl of 0.1 U/ml xanthine oxidase was added to 180 μl of 0.1 mol/l phosphate buffered saline (pH 7.2) containing 20 μl of 440 mmol/1 DMPO and 20 μl of 362 μmol/l xanthine so as to generate O₂·⁻. Then, the resulting mixture (200 μl) was transferred to a flat cell, and the generation of O₂·⁻ was examined by measuring the DMPO-OH spin adduct using the X-band ESR spin-trapping method. ESR measurement conditions will be described below.

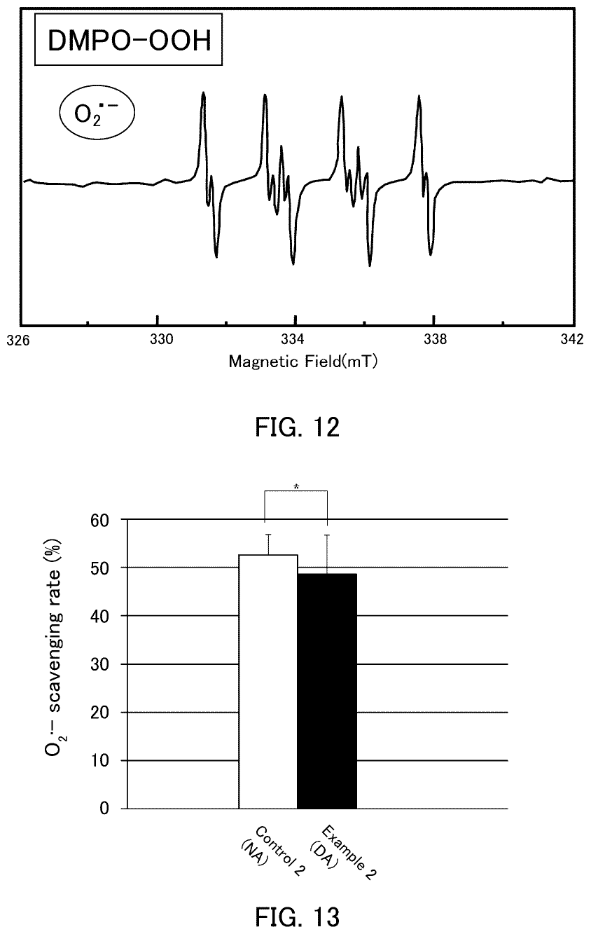

As indicated by Formula (A) below, the generated O₂·⁻ reacts with DMPO added to the reaction system to generate nitroxide (DMPO-OOH), which is a stable radical detectable by ESR. As shown in FIG. 12, this radical exhibits 12 absorption lines (peaks) derived from the internal magnetic field of ¹⁴N and ¹H at the β and γ positions in the ESR spectrum.

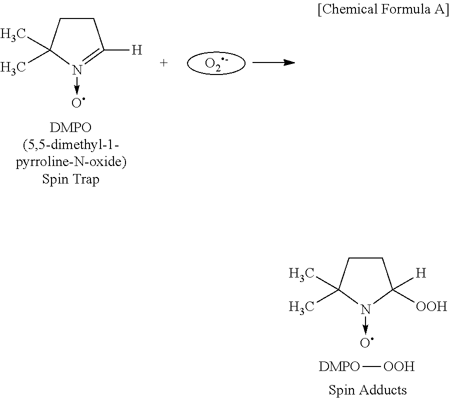

When a saliva sample is added to the reaction system, the intensity of signals obtained through ESR changes. The O₂·⁻ scavenging ability of the saliva sample was measured as follows. First, 20 μl of 0.1 U/ml XO was added to 180 μl of 0.1 mol/l phosphate buffered saline (pH 7.2) containing 20 μl of 440 mmol/l DMPO, 20 μl of 362 μmol/l xanthine, and 20 μl of the saliva sample so as to generate O₂·⁻. Then, the obtained mixture (200 μl) was transferred to a flat cell, and the O₂·⁻ scavenging ability of the saliva sample was measured by measuring the DMPO-OH spin adduct using the X-band ESR spin-trapping method.

In the ESR measurement, an electron spin resonance device (JES-RE 3X, X-band spectrometer, manufactured by JEOL Ltd.) was connected to WIN-RAD ESR data analyzer (Radical Research, Tokyo, Japan), and the following measurement conditions were used. The hyperfine coupling constant was calculated using the resonance frequency measured using a microwave frequency counter, and the resonance electric field measured using an electric field measuring device ES-FC5 (JEOL Ltd.). The detected spin adducts were quantified from the ESR spectra of manganese oxide standards. The actual measured signal intensity was expressed by a relative height normalized to the signal intensity of the ESR spectra of the manganese oxide standards. Also, the average for each group (Example 2 and Control 2) was calculated, and the O₂·⁻ scavenging ability was calculated as a relative value obtained using the reaction system to which a saliva sample was not added as the standard (100%). The results therefor are shown in FIG. 13.

### ESR Measurement Conditions

### Device:

Electron Spin Resonance (ESR) device (JES-RE 3X, X-band spectrometer, manufactured by JEOL Ltd.)

### Measurement Conditions:

Microwave power: 8.00 mW
Sweep time: 1 minute
Sweep width: 334.8±5 mT
Magnetic field modulation: 100 kHz 0.079 mT
Gain: ×400
Sweep time: 1 minute
Time constant: 0.03 seconds

FIG. 13 is a graph showing the ESR results for each saliva sample. In FIG. 13, the vertical axis indicates the relative value of O₂·⁻ scavenging rate, and the horizontal axis indicates the type of saliva sample. As shown in FIG. 13, the Down syndrome patient group (Example 2) had a significantly lower O₂·⁻ scavenging ability (SOD activity) as compared to the healthy subject group (Control 2).

Based on the above, it was found that the SOD activity decreased in the saliva of Down syndrome patients.

Because chromosome 21 is a trisomy in Down syndrome, it has been said that the SOD activity value increases. However, as shown in Example 2, it was found that the SOD activity decreased, as compared to the healthy subjects. Thus, it is presumed that in the Down syndrome patients, while the expression level of SOD increased and the apparent SOD activity value increased due to the trisomy of chromosome 21, in actuality, SOD in Down syndrome patients is denatured into SOD with weakened superoxide (O₂·⁻) scavenging ability. Note that the present invention is in no way limited to this presumption.

Further, it is known that Down syndrome patients are susceptible to dementia due to premature aging, and to exhibit mild cognitive impairment (MCI) symptoms. Therefore, it is conceivable that measurement of the SOD activity is also useful for diagnosis of MCI.

### Example 3

It was examined that the superoxide dismutase (SOD) activity decreased in the saliva derived from MCI patients.

Cognitive function test (Japanese version of MoCA: MoCA-J) was performed on 22 healthy subjects (aged 21 to 68 years) who gave informed consent in writing, and cognitive functions (visuospatial, executive function, nomenclature, memory, attention, repetition, word recall, abstraction, delayed recall, and orientation) were measured. Subjects with a MoCA-J score of 25 points or less were assessed as MCI patients (Example 3, n=8, average cognitive function 22.5), and subjects with a MoCA-J score of 26 points or more were assessed as healthy subjects (Control 3, n=14, average cognitive function 27.9). When selecting subjects, persons who had used a corticosteroid or immunosuppressant agent for a long period, persons who had a history of use of antibiotics within the past three months, and persons who had been treated with an antifungal agent within the past six weeks were excluded.

Then, saliva was collected, and the superoxide (O₂·⁻) scavenging ability (SOD activity) was measured in the same manner as that in Example 1 (2) and Example 2, except that the subjects of Example 3 (Example 3 and Control 3) were used instead of the subjects of Example 2 (Example 2 and Control 3). Four to six saliva samples were collected from each subject, and the O₂·⁻ scavenging ability of the multiple samples was measured, the average was calculated, and the calculated average was used as the O₂·⁻ scavenging ability of each subject.

The results therefor are summarized in Table. 2. As summarized in Table 2 below, the saliva of Example 3 had less than half the SOD activity as compared to the saliva of Control 3.

TABLE 2

| | Cognitive | | | |
|---|---|---|---|---|
| | function test | O₂^{•-} | Average | Average |
| Subject | results | suppression | cognitive | suppression |
| No. | (MCI) | ratio | function | ratio |
| 23 | 20 | 16.37% | 22.5 | 10.9% |
| 17 | 21 | 11.08% | (MCI group/Ex. 3) | |
| 8 | 22 | 21.74% | | |
| 10 | 22 | 13.39% | | |
| 9 | 23 | 2.36% | | |
| 13 | 23 | 17.55% | | |
| 1 | 24 | 3.46% | | |
| 5 | 25 | 1.22% | | |
| 6 | 26 | 28.67% | 27.9 | 21.8% |
| 16 | 26 | 33.01% | (Healthy subject | |
| 3 | 27 | 8.57% | group/Control 3) | |
| 14 | 27 | 50.04% | | |
| 21 | 27 | 16.52% | | |
| 22 | 27 | 28.77% | | |
| 7 | 28 | 17.34% | | |
| 15 | 28 | 11.08% | | |
| 2 | 29 | 8.43% | | |
| 11 | 29 | 21.16% | | |
| 12 | 29 | 3.97% | | |
| 18 | 29 | 10.05% | | |
| 19 | 29 | 7.98% | | |
| 20 | 29 | 59.83% | | |

Based on the above, it was found that, because the SOD activity decreased in the saliva of MCI patients, the SOD activity functions as an MCI marker.

From the above results, it was found that the radical scavenging ability functions as a physical frailty marker in Example 1. In Examples 2 and 3, it was found that the SOD activity functions as an MCI marker. From the above, it was proven that the mental frailty can be assessed by the combined use of the radical scavenging ability and the SOD activity.

While the present invention has been described above with reference to illustrative embodiments and examples, the present invention is by no means limited thereto. Various changes and modifications that may become apparent to those skilled in the art may be made in the configuration and specifics of the present invention without departing from the scope of the present invention.

This application claims priority from Japanese Patent Application No. 2021-133689 filed on Aug. 18, 2021. The entire subject matter of the Japanese Patent Application is incorporated herein by reference.

### Supplementary Notes

Some or all of the above embodiments and examples may be described as in the following Supplementary Notes, but are not limited thereto.

### Supplementary Note 1

A marker for detecting physical frailty, which is a radical.

### Supplementary Note 2

The marker according to Supplementary Note 1, which is a radical scavenging ability.

### Supplementary Note 3

The marker according to Supplementary Note 1 or 2, which is a radical in saliva.

### Supplementary Note 4

A test method for physical frailty, including:
measuring radical scavenging ability in a biological sample of a subject.

### Supplementary Note 5

The test method according to Supplementary Note 4, wherein in the measuring, a radical scavenging ability value is measured.

### Supplementary Note 6

The test method according to Supplementary Note 5, including:
testing likelihood of the subject having physical frailty by comparing the radical scavenging ability value in the biological sample of the subject with a threshold, wherein the threshold is a threshold calculated from radical scavenging ability values in biological samples of healthy subjects, radical scavenging ability values in biological samples of physical frailty patients, or the radical scavenging ability values in the biological samples of the healthy subjects and the physical frailty patients.

### Supplementary Note 7

The test method according to Supplementary Note 6, wherein
in the testing, when the radical scavenging ability value in the biological sample of the subject is higher than the radical scavenging ability values in the biological samples of the healthy subjects, when the radical scavenging ability value in the biological sample of the subject is the same as the radical scavenging ability values in the biological samples of the physical frailty patients, when the radical scavenging ability value in the biological sample of the subject is higher than the radical scavenging ability values in the biological samples of the physical frailty patients, and/or when the radical scavenging ability value in the biological sample of the subject is higher than the threshold calculated from the radical scavenging ability values in the biological samples of the healthy subjects and the physical frailty patients, the subject is assessed as having physical frailty.

### Supplementary Note 8

The test method according to any one of Supplementary Notes 4 to 7, wherein
the radical scavenging ability is a lipid radical scavenging ability.

### Supplementary Note 9

The test method according to any one of Supplementary Notes 4 to 8, wherein the biological sample is saliva.

### Supplementary Note 10

The test method according to any one of Supplementary Notes 4 to 9, wherein
the testing for physical frailty is detection of physical frailty, determination of physical frailty, screening for physical frailty, determination of a physical frailty preventive effect, determination of a physical frailty therapeutic effect, determination of a physical frailty patient who responds to a therapeutic agent, determination of a therapeutic agent that takes an effect on an individual physical frailty patient, a test method for diagnosing physical frailty, or a test for treating physical frailty.

### Supplementary Note 11

The test method according to any one of Supplementary Notes 4 to 10, wherein the subject is a mild cognitive impairment (MCI) patient, and
the physical frailty is mental frailty.

### Supplementary Note 12

The method according to any one of Supplementary Notes 4 to 11, further including: administering a therapeutic agent for physical frailty to a subject who has received a test result indicating that the subject has physical frailty.

### Supplementary Note 13

A test reagent for physical frailty, including:
a reagent for measuring radical scavenging ability.

### Supplementary Note 14

The test reagent according to Supplementary Note 13, including:
a radical generating agent; and
a radical detecting agent.

### Supplementary Note 15

The test reagent according to Supplementary Note 14, wherein
the radical generating agent includes a lipid radical generating agent, and
the radical detecting agent includes a lipid radical detecting agent.

### Supplementary Note 16

The test reagent according to any one of Supplementary Notes 13 to 15 for use in the test method according to any one of Supplementary Notes 4 to 12.

### Supplementary Note 17

A method for screening a therapeutic candidate substance for physical frailty, including:
selecting, from test substances, an activating substance that reduces radical scavenging ability as a therapeutic candidate substance for physical frailty.

### Supplementary Note 18

The screening method according to Supplementary Note 17, including:
causing each of the test substances to be also present in a radical generation system, and measuring radical scavenging ability; and
choosing the test substance as the therapeutic candidate substance when the radical scavenging ability obtained in the causing of each of the test substances to be also present in a radical generation system, and measuring radical scavenging ability is lower than that in a control system in which the test substance is not present.

### Supplementary Note 19

The screening method according to Supplementary Note 17 or 18, wherein
the test substance is at least one selected from the group consisting of a low-molecular-weight compound, a peptide, a protein, and a nucleic acid.

### Supplementary Note 20

A detection method for radical scavenging ability in a subject who has a suspected case of physical frailty, including:
detecting radical scavenging ability in a biological sample of the subject using a reagent for measuring radical scavenging ability.

### Supplementary Note 21

The detection method according to Supplementary Note 20, wherein
the biological sample is saliva.

### Supplementary Note 22

Use of a reagent for measuring radical scavenging ability in order to test likelihood of physical frailty.

### Supplementary Note 23

A test method for mental frailty, including:
measuring radical scavenging ability in a biological sample of a subject (first measuring); and
measuring superoxide dismutase (SOD) activity in the biological sample of the subject (second measuring).

### Supplementary Note 24

The test method according to Supplementary Note 23, wherein
in the first measuring, a radical scavenging ability value is measured, and
in the second measuring, an SOD activity value is measured.

### Supplementary Note 25

The test method according to Supplementary Note 24, including:
testing likelihood of the subject having mental frailty by comparing the radical scavenging ability value in the biological sample of the subject with a first threshold and comparing the SOD activity value in the biological sample of the subject with a second threshold, wherein
   the first threshold is a threshold calculated from radical scavenging ability values in biological samples of healthy subjects, radical scavenging ability values in biological samples of physical frailty patients, radical scavenging ability values in biological samples of mental frailty patients, or the radical scavenging ability values in the biological samples of the healthy subjects and the physical frailty patients or the mental frailty patients, and
   the second threshold is a threshold calculated from SOD activity values in biological samples of healthy subjects, SOD activity values in biological samples of MCI patients, SOD activity values in biological samples of mental frailty patients, or the SOD activity values in the biological samples of the healthy subjects and the MCI patients or the mental frailty patients.

### Supplementary Note 26

The test method according to Supplementary Note 25, wherein
in the testing,
when the radical scavenging ability value in the biological sample of the subject is higher than the radical scavenging ability values in the biological samples of the healthy subjects, when the radical scavenging ability value in the biological sample of the subject is the same as the radical scavenging ability values in the biological samples of the physical frailty patients or the mental frailty patients, when the radical scavenging ability value in the biological sample of the subject is higher than the radical scavenging ability values in the biological samples of the physical frailty patients or the mental frailty patients, and/or when the radical scavenging ability value in the biological sample of the subject is higher than the threshold calculated from the radical scavenging ability values in the biological samples of the physical frailty patients or the mental frailty patients, and
when the SOD activity value in the biological sample of the subject is lower than the SOD activity values in the biological samples of the healthy subjects, when the SOD activity value in the biological sample of the subject is the same as the SOD activity values in the biological samples of the MCI patients or the mental frailty patients, and/or when the SOD activity value in the biological sample of the subject is lower than the SOD activity values in the biological samples of the MCI patients or the mental frailty patients,
the subject is assessed as having mental frailty.

### Supplementary Note 27

The test method according to any one of Supplementary Notes 23 to 26, wherein
the radical scavenging ability is lipid radical scavenging ability and/or the SOD activity is superoxide scavenging activity.

### Supplementary Note 28

The test method according to any one of Supplementary Notes 23 to 27, wherein
the biological sample is saliva.

### Supplementary Note 29

The test method according to any one of Supplementary Notes 23 to 28, wherein
the testing for mental frailty is detection of mental frailty, determination of mental frailty, screening for mental frailty, determination of a mental frailty preventive effect, determination of a mental frailty therapeutic effect, determination of a mental frailty patient who responds to a therapeutic agent, determination of a therapeutic agent that takes an effect on an individual mental frailty patient, a test method for diagnosing mental frailty, or a test for treating mental frailty.

### Supplementary Note 30

A test kit for mental frailty, including:
a reagent for measuring radical scavenging ability; and
a reagent for measuring SOD activity.

### Supplementary Note 31

The test kit according to Supplementary Note 30, wherein
the reagent for measuring radical scavenging ability contains a radical generating agent and a radical detecting agent, and/or
the reagent for measuring SOD activity contains a probe for detecting xanthine, xanthine oxidase, and superoxide.

### Supplementary Note 32

The test kit according to Supplementary Note 30 or 31, wherein the radical is a lipid radical.

### Supplementary Note 33

The test kit according to any one of Supplementary Notes 30 to 32 for use in the test method according to any one of Supplementary Notes 23 to 29.

### Supplementary Note 34

A method for screening a therapeutic candidate substance for mental frailty, including:
selecting, from test substances, an activating substance that reduces radical scavenging ability and improves SOD activity as a therapeutic candidate substance for mental frailty.

### Supplementary Note 35

The screening method according to Supplementary Note 34, including:
causing each of the test substances to be also present in a radical generation system, and measuring radical scavenging ability (first measuring);
causing each of the test substances to be also present in a system containing superoxide and SOD, and measuring SOD activity (second measuring); and
choosing the test substance as the therapeutic candidate substance when the radical scavenging ability obtained in the first measuring is lower than that in a control system in which the test substance is not present and when the SOD activity obtained in the second measuring is higher than that in a control system in which the test substance is not present.

### Supplementary Note 36

The screening method according to Supplementary Note 34 or 35, wherein
the test substance is at least one selected from the group consisting of a low-molecular-weight compound, a peptide, a protein, and a nucleic acid.

### Supplementary Note 37

A detection method for radical scavenging ability and SOD activity in a subject who has a suspected case of mental frailty, including:
detecting radical scavenging ability in a biological sample of the subject using a reagent for measuring radical scavenging ability (first detecting); and
detecting SOD activity in the biological sample of the subject using a reagent for measuring SOD activity (second detecting).

### Supplementary Note 38

The detection method according to Supplementary Note 37, wherein the biological sample is saliva.

### Supplementary Note 39

Use of a reagent for measuring radical scavenging ability in order to test the likelihood of mental frailty.

### INDUSTRIAL APPLICABILITY

As described above, according to the present invention, the likelihood of subjects having physical frailty (morbidity risk) and the like can be tested by measuring the radical scavenging ability. Also, in the present invention, because the radical scavenging ability serves as a target with respect to physical frailty, therapeutic candidate substances for physical frailty can be obtained through screening using the target. Therefore, the present invention is extremely useful in the clinical and biochemical fields.

1. A test method for physical frailty, comprising:
measuring radical scavenging ability in a biological sample of a subject.

2. The test method according to claim 1, wherein
in the measuring, a radical scavenging ability value is measured.

3. The test method according to claim 2, comprising:
testing the likelihood of the subject having physical frailty by comparing the radical scavenging ability value in the biological sample of the subject with a threshold, wherein
the threshold is a threshold calculated from radical scavenging ability values in biological samples of healthy subjects, radical scavenging ability values in biological samples of physical frailty patients, or the radical scavenging ability values in the biological samples of the healthy subjects and the physical frailty patients.

4. The test method according to claim 3, wherein
in the testing, when the radical scavenging ability value in the biological sample of the subject is higher than the radical scavenging ability values in the biological samples of the healthy subjects, when the radical scavenging ability value in the biological sample of the subject is the same as the radical scavenging ability values in the biological samples of the physical frailty patients, when the radical scavenging ability value in the biological sample of the subject is higher than the radical scavenging ability values in the biological samples of the physical frailty patients, and/or when the radical scavenging ability value in the biological sample of the subject is higher than the threshold calculated from the radical scavenging ability values in the biological samples of the healthy subjects and the physical frailty patients, the subject is assessed as having physical frailty.

5. The test method according to claim 1, wherein
the radical scavenging ability is lipid radical scavenging ability.

6. The test method according to claim 1, wherein
the biological sample is saliva.

7. The test method according to claim 1, wherein
the testing for physical frailty is detection of physical frailty, determination of physical frailty, screening for physical frailty, determination of a physical frailty preventive effect, determination of a physical frailty therapeutic effect, determination of a physical frailty patient who responds to a therapeutic agent, determination of a therapeutic agent that takes an effect on an individual physical frailty patient, a test method for diagnosing physical frailty, or a test for treating physical frailty.

8. The test method according to claim 1, wherein
the subject is a mild cognitive impairment (MCI) patient, and
the physical frailty is mental frailty.

9. A test reagent for physical frailty, comprising:
a reagent for measuring radical scavenging ability.

10. The test reagent according to claim 9, comprising:
a radical generating agent; and
a radical detecting agent.

11. The test reagent according to claim 10, wherein
the radical generating agent includes a lipid radical generating agent, and
the radical detecting agent includes a lipid radical detecting agent.

12. The test reagent according to claim 9 for use in a test method for physical frailty, wherein,
the test method comprises:
measuring radical scavenging ability in a biological sample of a subject.

13-17. (canceled)

18. A test method for mental frailty, comprising:
measuring radical scavenging ability in a biological sample of a subject (first measuring); and
measuring superoxide dismutase (SOD) activity in the biological sample of the subject (second measuring).

19. The test method according to claim 18, wherein
in the first measuring, a radical scavenging ability value is measured, and
in the second measuring, an SOD activity value is measured.

20. The test method according to claim 19, comprising:
testing likelihood of the subject having mental frailty by comparing the radical scavenging ability value in the biological sample of the subject with a first threshold and comparing the SOD activity value in the biological sample of the subject with a second threshold, wherein
the first threshold is a threshold calculated from radical scavenging ability values in biological samples of healthy subjects, radical scavenging ability values in biological samples of physical frailty patients, radical scavenging ability values in biological samples of mental frailty patients, or the radical scavenging ability values in the biological samples of the healthy subjects and the physical frailty patients or the mental frailty patients, and
the second threshold is a threshold calculated from SOD activity values in biological samples of healthy subjects, SOD activity values in biological samples of MCI patients, SOD activity values in biological samples of mental frailty patients, or the SOD activity values in the biological samples of the healthy subjects and the MCI patients or the mental frailty patients.

21. The test method according to claim 20, wherein
in the testing,
when the radical scavenging ability value in the biological sample of the subject is higher than the radical scavenging ability values in the biological samples of the healthy subjects, when the radical scavenging ability value in the biological sample of the subject is the same as the radical scavenging ability values in the biological samples of the physical frailty patients or the mental frailty patients, when the radical scavenging ability value in the biological sample of the subject is higher than the radical scavenging ability values in the biological samples of the physical frailty patients or the mental frailty patients, and/or when the radical scavenging ability value in the biological sample of the subject is higher than the first threshold calculated from the radical scavenging ability values in the biological samples of the physical frailty patients or the mental frailty patients, and
when the SOD activity value in the biological sample of the subject is lower than the SOD activity values in the biological samples of the healthy subjects, when the SOD activity value in the biological sample of the subject is the same as the SOD activity values in the biological samples of the MCI patients or the mental frailty patients, when the SOD activity value in the biological sample of the subject is lower than the SOD activity values in the biological samples of the MCI patients or the mental frailty patients, and/or when the SOD activity value in the biological sample of the subject is lower than the second threshold calculated from the SOD activity values in the biological samples of the healthy subjects and the mental frailty patients or the MCI patients,
the subject is assessed as having mental frailty.

22. The test method according to claim 18, wherein
the radical scavenging ability is lipid radical scavenging ability and/or the SOD activity is superoxide scavenging activity.

23. The test method according to claim 18, wherein
the biological sample is saliva.

24. The test method according to claim 18, wherein
the testing for mental frailty is detection of mental frailty, determination of mental frailty, screening for mental frailty, determination of a mental frailty preventive effect, determination of a mental frailty therapeutic effect, determination of a mental frailty patient who responds to a therapeutic agent, determination of a therapeutic agent that takes an effect on an individual mental frailty patient, a test method for diagnosing mental frailty, or a test for treating mental frailty.

25-33. (canceled)


SEQUENCE LISTING (2 sequences)

SEQ ID NO: 1 - DNA, 895 bp, Homo sapiens
gcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaa

SEQ ID NO: 2 - protein, 154 aa, Homo sapiens [sequence omitted]